(19) United States
(12) Patent Application Publication
(10) Pub. No.: US 2006/0293320 A1
(43) Pub. Date: Dec. 28, 2006

(54) Heteroaryl derivatives for treating viruses

(75) Inventors: Franz Ulrich Schmitz, Mill Valley, CA (US); Janos Botyanszki, Fremont, CA (US); Christopher Don Roberts, Belmont, CA (US)

Correspondence Address: FOLEY & LARDNER LLP, 1530 PAGE MILL ROAD, PALO ALTO, CA, 94304, US

(73) Assignee: Genelabs Technologies, Inc.

(21) Appl. No.: 11/473,511

(22) Filed: Jun. 22, 2006

Related U.S. Application Data
(60) Provisional application No. 60/693,700, filed on Jun. 24, 2005.

(51) Int. Cl. A61K 31/5377; A61K 31/497; A61K 31/4709; C07D 413/14; C07D 417/14

(52) U.S. Cl. 514/233.2; 514/255.05; 514/314; 514/311; 544/405; 546/167; 544/127

(57) ABSTRACT

Disclosed are compounds, compositions, and methods for treating Flaviviridae family virus infections.

### CROSS-REFERENCE TO RELATED APPLICATION

This application claims the benefit under 35 U.S.C. 119(e) to co-pending provisional application U.S. Ser. No. 60/693,700 filed on Jun. 24, 2005, which is incorporated herein by reference in its entirety.

### BACKGROUND OF THE INVENTION

### Field of the Invention

The invention relates to the field of pharmaceutical chemistry, in particular to compounds, compositions and methods for treating viral infections in mammals mediated, at least in part, by a virus in the Flaviviridae family of viruses.

### References

The following publications are cited in this application as superscript numbers:
1. Szabo, E. et al., Pathol. Oncol. Res. 2003, 9:215-221.
2. Hoofnagle J. H., Hepatology 1997, 26:15S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem 39, 223-237, 2004.
9. Watashi, K. et al., Molecular Cell, 19, 111-122, 2005
10. Horsmans, Y. et al., Hepatology, 42, 724-731, 2005

All of the above publications are herein incorporated by reference in their entirety to the same extent as if each individual publication was specifically and individually indicated to be incorporated by reference in its entirety.

### State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

The present invention is directed to novel compounds, compositions, and methods for treating of viral infections in mammals mediated, at least in part, by a member of the Flaviviridae family viruses such as HCV. Specifically, compounds of this invention are represented by formula (1): 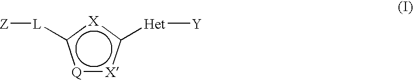
wherein:

L is selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;

one of X or X′ is N—R¹ and the other is selected from the group consisting of C—R², N, O or S;

Q is selected from the group consisting of C—R, N, O or S with the proviso that when X or X′ is O or S, then Q is selected from C—R and N;

R is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl; R¹ and R² are independently selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CH₂CONR³R⁴, and —NR³R⁴; where each of R^{1a}, R³ and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl;

Z is selected from the group consisting of:

(a) hydrogen, halo, alkyl, substituted alkyl, alkenyl, substituted alkenyl, alkoxy, substituted alkoxy, cyano, aryl, substituted aryl, heteroaryl, substituted heteroaryl, amino, and substituted amino;

(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;

(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic, and substituted heterocyclic or, alternatively, R⁵ and R⁶ together with the nitrogen atom pendent thereto, form a heterocyclic, a substituted heterocyclic, a heteroaryl or a substituted heteroaryl ring group;

(d) —C(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each R⁷, R¹⁰ and R¹¹ is independently hydrogen, alkyl, substituted alkyl, cycloalkyl, or substituted cycloalkyl, and wherein each R⁷ and R¹⁰ is optionally substituted with at least one halo, hydroxy, carboxy, carboxy ester, alkyl, alkoxy, amino, substituted amino; or alternatively, R⁷ and R¹⁰ or R¹⁰ and R¹¹ together with the atoms bound thereto join together to form an optionally substituted heterocyclic group;

(e) —C(X³)—N(R¹²)CR¹³R¹³C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above;

R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or, alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;
R¹² is selected from hydrogen and alkyl or, when R¹³ and R^{13′} are not taken together to form a ring and when R¹³ or R^{13′} and R¹⁰ or R¹¹ are not joined to form a heterocyclic or substituted heterocyclic group, then R¹², together with the nitrogen atom pendent thereto, may be taken together with one of R¹³ and R^{13′} to form a heterocyclic or substituted heterocyclic ring group;

(f) —C(X²)—N(R¹²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and

(g) carboxylic acid isostere;

with the proviso that when L is a bond, Z is not hydrogen;

Het is selected from the group consisting of arylene, substituted arylene, heteroarylene and substituted heteroarylene; and

Y is selected from the group consisting of alkyl, aryl, heteroaryl, substituted aryl, and substituted heteroaryl;

or a pharmaceutically acceptable salt, ester, stereoisomer, prodrug, or tautomer thereof.

### DETAILED DESCRIPTION OF THE INVENTION

The invention is directed to compounds, compositions and methods for treating Flaviviridae family viral infections.

In one embodiment, the present invention provides compounds represented by formula (I): 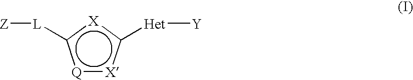
wherein:

L is selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;

one of X or X′ is N—R¹ and the other is selected from the group consisting of C—R², N, O or S;

Q is selected from the group consisting of C—R, N, O or S with the proviso that when X or X′ is O or S, then Q is selected from C—R and N;

R is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl;

R¹ and R² are independently selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CH₂CONR³R⁴, and —NR³R⁴; where each of R^{1a}, R³ and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl;

Z is selected from the group consisting of:

(a) hydrogen, halo, alkyl, substituted alkyl, alkenyl, substituted alkenyl, alkoxy, substituted alkoxy, cyano, aryl, substituted aryl, heteroaryl, substituted heteroaryl, amino, and substituted amino;

(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;

(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic, and substituted heterocyclic or, alternatively, R⁵ and R⁶ together with the nitrogen atom pendent thereto, form a heterocyclic, a substituted heterocyclic, a heteroaryl or a substituted heteroaryl ring group;

(d) —C(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each R⁷, R¹⁰ and R¹¹ is independently hydrogen, alkyl, substituted alkyl, cycloalkyl, or substituted cycloalkyl, and wherein each R⁷ and R¹⁰ is optionally substituted with at least one halo, hydroxy, carboxy, carboxy ester, alkyl, alkoxy, amino, substituted amino; or alternatively, R⁷ and R¹⁰ or R¹⁰ and R¹¹ together with the atoms bound thereto join together to form an optionally substituted heterocyclic group;

(e) —C(X³)—N(R¹²)CR¹³R^{13′}C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above; R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or, alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;

R¹² is selected from hydrogen and alkyl or, when R¹³ and R^{13′} are not taken together to form a ring and when R¹³ or R^{13′} and R¹⁰ or R¹¹ are not joined to form a heterocyclic or substituted heterocyclic group, then R¹², together with the nitrogen atom pendent thereto, may be taken together with one of R¹³ and R^{13′} to form a heterocyclic or substituted heterocyclic ring group;

(f) —C(X²)—N(R¹²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and

(g) carboxylic acid isostere;

with the proviso that when L is a bond, Z is not hydrogen;

Het is selected from the group consisting of arylene, substituted arylene, heteroarylene and substituted heteroarylene; and

Y is selected from the group consisting of alkyl, aryl, heteroaryl, substituted aryl, and substituted heteroaryl;

or a pharmaceutically acceptable salt, ester, stereoisomer, prodrug, or tautomer thereof.

In other embodiments, the present invention is directed to compounds of formula (I) having formulae (II), (III), and (IV) or the pharmaceutically acceptable salt, ester, stereoisomer, prodrug, or tautomer thereof: 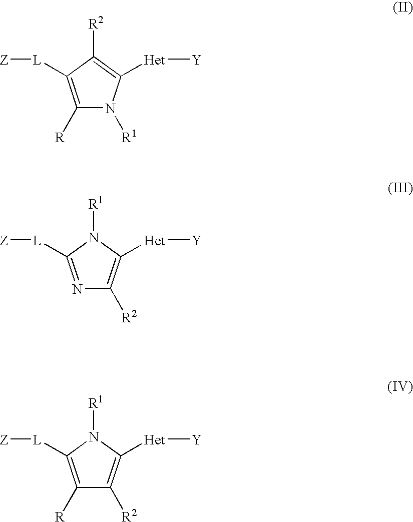
wherein Z, L, R, R¹, R², Het, and Y are previously defined for formula (I).

In another embodiment, the present invention provides compounds of formula (V) or a pharmaceutically acceptable salt, ester, stereoisomer, prodrug, or tautomer thereof:

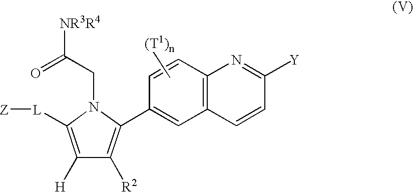

where Z, L, R², R³, R⁴, and Y are previously defined; T¹ is selected from the group consisting of alkyl, substituted alkyl, alkoxy, substituted alkoxy, amino, substituted amino, cyano, carboxyl, carboxyl ester, halo, hydroxy, heterocyclic, substituted hetereocyclic, and nitro; and n is an integer equal to 0, 1, or 2.

In some preferred embodiments, the invention provides compounds of formula (I)-(IV) where R is hydrogen, halo, or methyl.

In some preferred embodiments, the invention provides compounds of formula (I)-(V) where Z is —COOH, —COOR^{z} (where R^{z} is as defined above), 1H-tetrazol-5-yl, —C(O)NHSO₂CF₃,

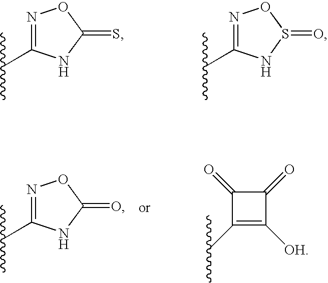

In other preferred embodiments, the invention provides compounds of formula (I)-(V) where L is a bond.

In yet other preferred embodiments, the invention provides compounds of formula (I)-(V) where L is —CH═CH— or —(CH₃)C═CH—, each having either a cis or trans orientation.

In some embodiments, the invention provides compounds of formula (I)-(V) where L is a heteroarylene or a substituted heteroarylene. In some such embodiments, Z-L- form a group having the formula: 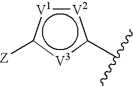
where V¹, V², and V³ are independently selected from the group consisting of O, S, N, NH, or CH. In some aspects Z is COOH. In other aspects, V¹, V², and V³ have one of the following combinations:

V¹ is CH, V² is NH, and V³ is CH;

V¹ is NH, V² is CH, and V³ is CH;

V¹ is CH, V² is CH, and V³ is N;

V¹ is CH, V² is NH, and V³ is N;

V¹ is NH, V² is CH, and V³ is N;

V¹ is NH, V² is N, and V³ is CH;

V¹ is NH, V² is N, and V³ is N;

V¹ is CH, V² is O, and V³ is CH;

V¹ is CH, V² is CH, and V³ is O;

V¹ is CH, V² is S, and V³ is CH;

V¹ is CH, V² is CH, and V³ is S;

V¹ is CH, V² is O, and V³ is N;

V¹ is CH, V² is O, and V³ is N;

V¹ is CH, V² is N, and V³ is O;

V¹ is CH, V² is S, and V³ is N; or

V¹ is CH, V² is N, V³ is S.

In still other preferred embodiments, the invention provides compounds of formula (I)-(V) where Het is heteroarylene or substituted heteroarylene, Y is aryl, heteroaryl, substituted aryl, or substituted heteroaryl, and Het and Y together form a -Het-Y group. In some embodiments of the invention, -Het-Y group has the formula (H1)

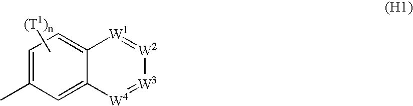

where each of W¹, W², W³ and W⁴ is independently selected from N, CH, CT², and C—Y, provided that no more than 2 of W¹, W², W³ and W⁴ are N; provided that one of W¹, W², W³ and W⁴ is C—Y; and further provided wherein no more than one N in the ring system is optionally oxidized to form the N-oxide. T¹ and T² are independently selected from the group consisting of alkyl, substituted alkyl, alkoxy, substituted alkoxy, amino, substituted amino, cyano, carboxyl, carboxyl ester, halo, hydroxy, heterocyclic, substituted hetereocyclic, and nitro; and n is an integer equal to 0, 1, or 2. In other preferred embodiments, said -Het-Y group has the formula (H2) 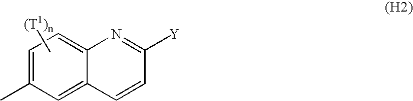
where T¹, n, and Y are defined as for formula (H1).

In some preferred embodiments, the invention provides compounds of formula (I)-(V) where Y is heteroaryl or substituted heteroaryl. In other preferred embodiments, Y is thiazole-5-yl or 2,4-dimethylthiazol-5-yl.

In some preferred embodiments, the invention provides compounds of formula (I)-(V) where the -Het-Y group is

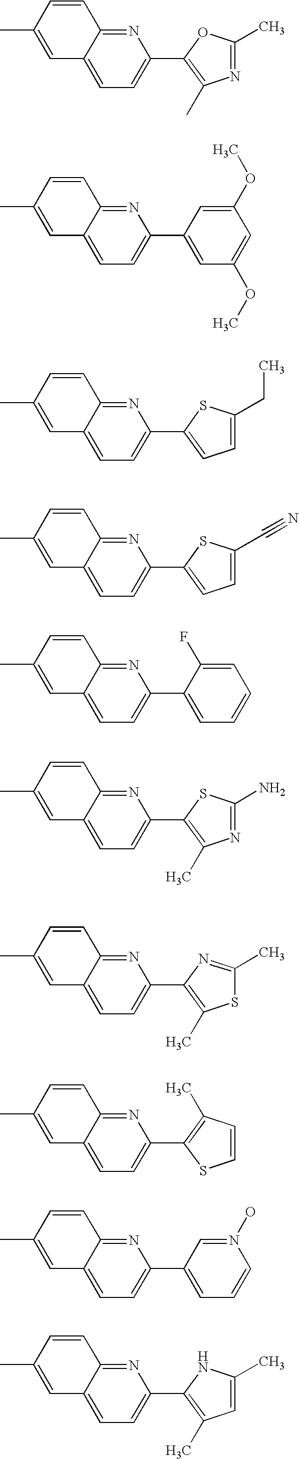

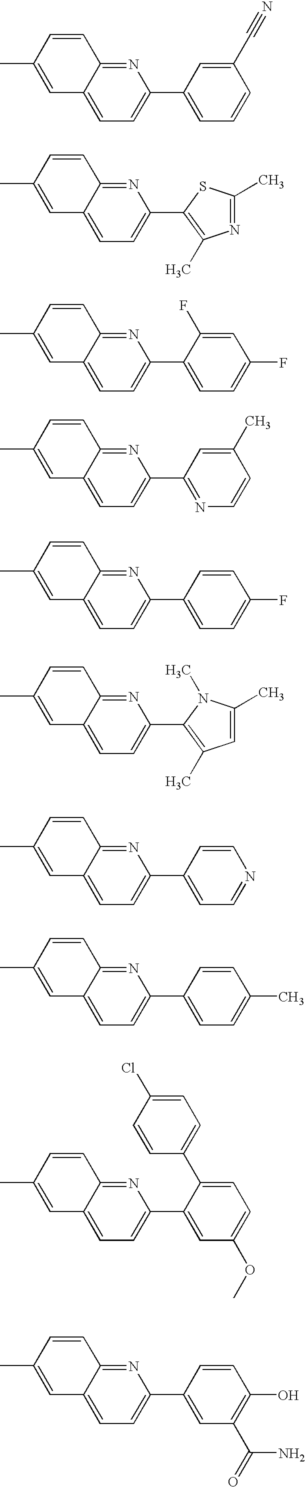

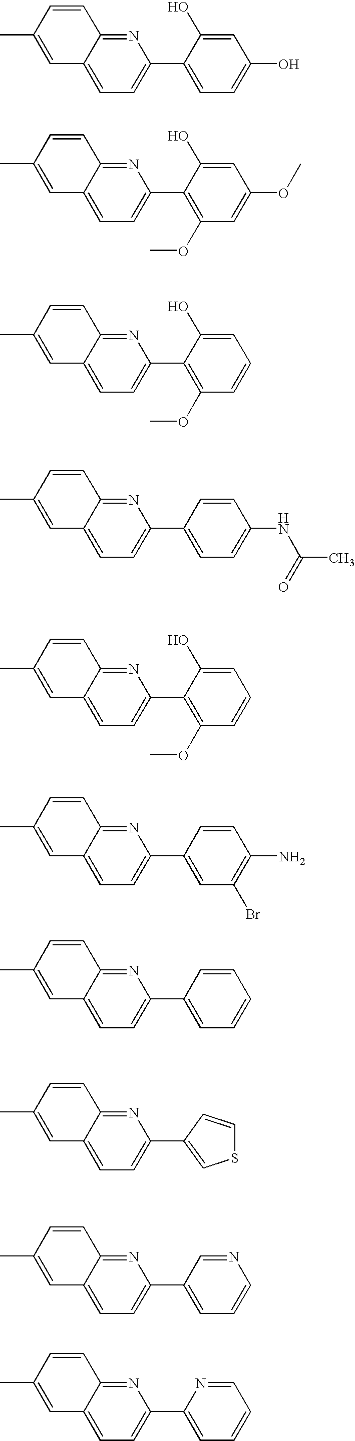

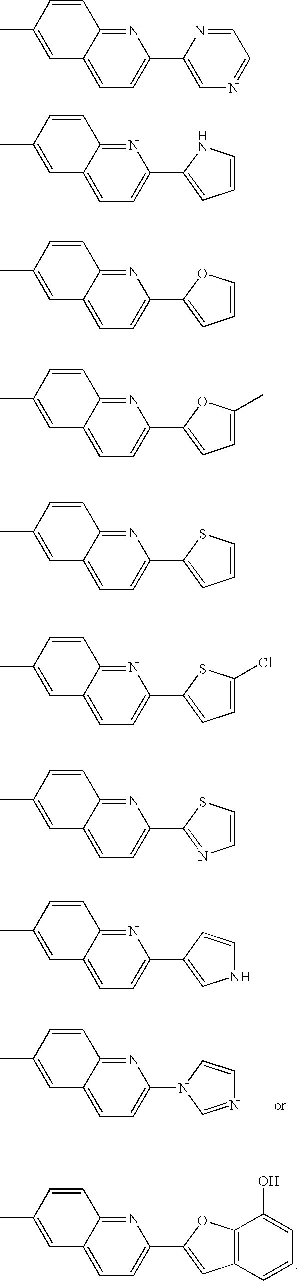

In some preferred embodiments, the invention provides compounds of formula (I)-(V) where R¹ or R² is selected from the group consisting of —COOH, —CH₂COOR^{1a}, and —CH₂CONR³R⁴ when said R¹ or R² is attached to a ring atom adjacent to a ring atom bearing L. In other embodiments, R³ and R⁴, together with the nitrogen to which they are attached, form a morpholino ring.

In some preferred embodiments, the invention provides compounds of formula (I)-(V) where R¹ or R² is cyclohexyl when said R¹ or R² is attached to a ring atom adjacent to a ring atom bearing R.

The present invention further provides compounds resulting from a combination of any of the variables relating to the atoms and substituents of formula (I)-(V), particularly those variables in the preferred embodiments above. Preferred compounds of this invention resulting form such combinations include, by way of example, those set forth in Table I below and their pharmaceutically acceptable salt, ester, stereoisomer, prodrug, or tautomer thereof.

TABLE I

| Cmpd. | Structure | Name |
|---|---|---|
| 1 | 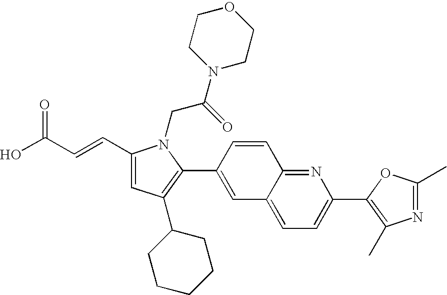 | (E)-3-(4-cyclohexyl-5-(2-(2,4-dimethyloxazol-5- yl)quinolin-6-yl)-1-(2-morpholino-2-oxoethyl)- 1H-pyrrol-2-yl)acrylic acid |
| 2 | 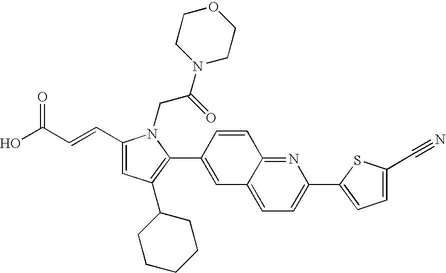 | (E)-3-(5-(2-(5-cyanothiophen-2-yl)quinolin-6- yl)-4-cyclohexyl-1-(2-morpholino-2-oxoethyl)- 1H-pyrrol-2-yl)acrylic acid |
| 3 | 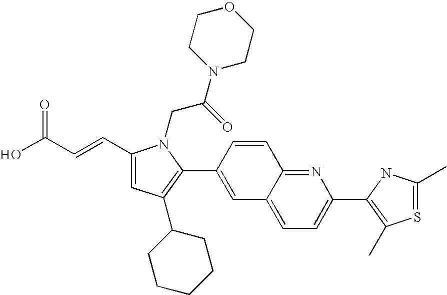 | (E)-3-(4-cyclohexyl-5-(2-(2,5-dimethylthiazol-4- yl)quinolin-6-yl)-1-(2-morpholino-2-oxoethyl)- 1H-pyrrol-2-yl)acrylic acid |
| 4 | 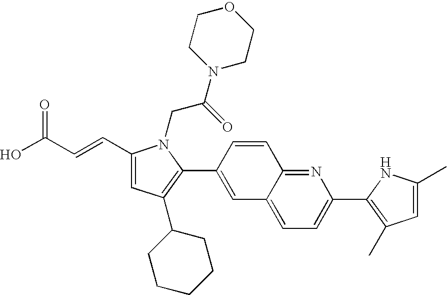 | (E)-3-(4-cyclohexyl-5-(2-(3,5-dimethyl-1H- pyrrol-2-yl)quinolin-6-yl)-1-(2-morpholino-2- oxoethyl)-1H-pyrrol-2-yl)acrylic acid |
| 5 | 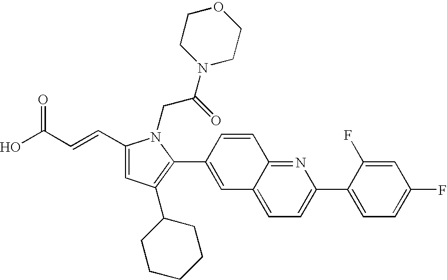 | (E)-3-(4-cyclohexyl-5-(2-(2,4- difluorophenyl)quinolin-6-yl)-1-(2-morpholino- 2-oxoethyl)-1H-pyrrol-2-yl)acrylic acid |
| 6 | 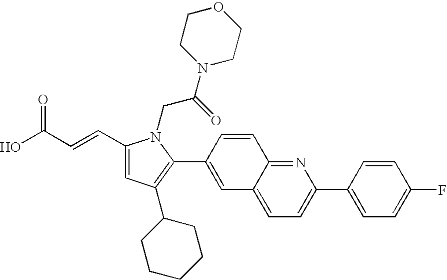 | (E)-3-(4-cyclohexyl-5-(2-(4- fluorophenyl)quinolin-6-yl)-1-(2-morpholino-2- oxoethyl)-1H-pyrrol-2-yl)acrylic acid |
| 7 | 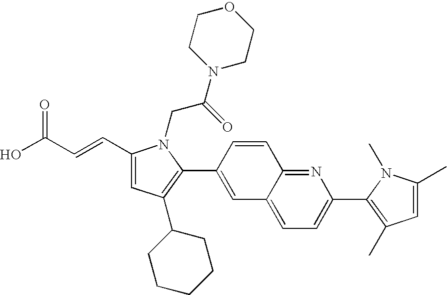 | (E)-3-(4-cyclohexyl-5-(2-(1,3,5-trimethyl-1H- pyrrol-2-yl)quinolin-6-yl)-1-(2-morpholino-2- oxoethyl)-1H-pyrrol-2-yl)acrylic acid |
| 8 | 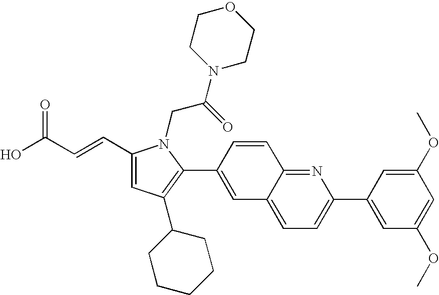 | (E)-3-(4-cyclohexyl-5-(2-(3,5- dimethoxyphenyl)quinolin-6-yl)-1-(2- morpholino-2-oxoethyl)-1H-pyrrol-2-yl)acrylic acid |
| 9 | 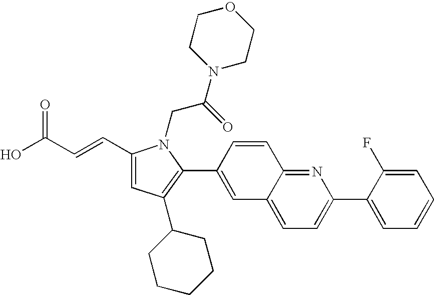 | (E)-3-(4-cyclohexyl-5-(2-(2- fluorophenyl)quinolin-6-yl)-1-(2-morpholino-2- oxoethyl)-1H-pyrrol-2-yl)acrylic acid |
| 10 | 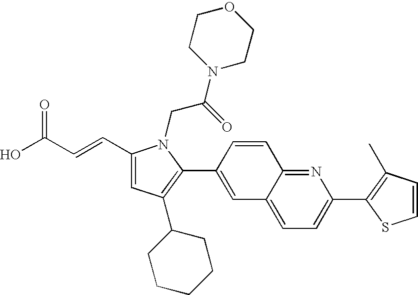 | (E)-3-(4-cyclohexyl-5-(2-(3-methylthiophen-2- yl)quinolin-6-yl)-1-(2-morpholino-2-oxoethyl)- 1H-pyrrol-2-yl)acrylic acid |
| 11 | 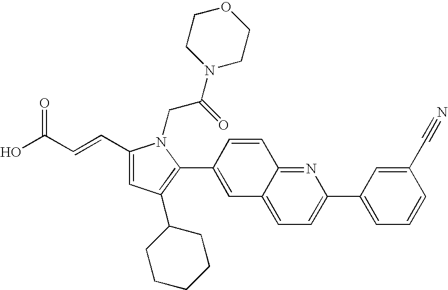 | (E)-3-(5-(2-(3-cyanophenyl)quinolin-6-yl)-4- cyclohexyl-1-(2-morpholino-2-oxoethyl)-1H- pyrrol-2-yl)acrylic acid |
| 12 | 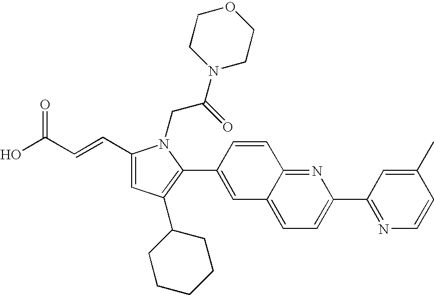 | (E)-3-(4-cyclohexyl-5-(2-(4-methylpyridin-2- yl)quinolin-6-yl)-1-(2-morpholino-2-oxoethyl)- 1H-pyrrol-2-yl)acrylic acid |
| 13 | 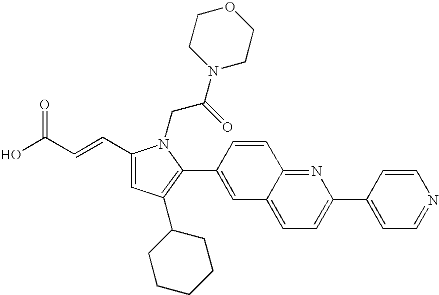 | (E)-3-(4-cyclohexyl-1-(2-morpholino-2- oxoethyl)-5-(2-(pyridin-4-yl)quinolin-6-yl)-1H- pyrrol-2-yl)acrylic acid |
| 14 | 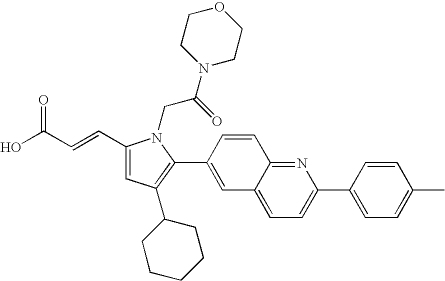 | (E)-3-(4-cyclohexyl-1-(2-morpholino-2- oxoethyl)-5-(2-p-tolylquinolin-6-yl)-1H-pyrrol- 2-yl)acrylic acid |
| 15 | 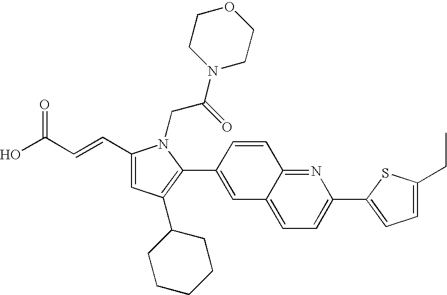 | (E)-3-(4-cyclohexyl-5-(2-(5-ethylthiophen-2- yl)quinolin-6-yl)-1-(2-morpholino-2-oxoethyl)- 1H-pyrrol-2-yl)acrylic acid |
| 16 | 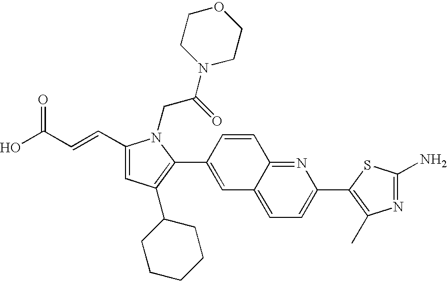 | (E)-3-(5-(2-(2-amino-4-methylthiazol-5- yl)quinolin-6-yl)-4-cyclohexyl-1-(2-morpholino- 2-oxoethyl)-1H-pyrrol-2-yl)acrylic acid |
| 17 | 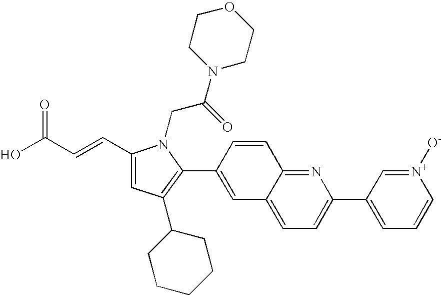 | (E)-3-(4-cyclohexyl-1-(2-morpholino-2- oxoethyl)-5-(2-(N-oxo-pyridin-3-yl)quinolin-6- yl)-1H-pyrrol-2-yl)acrylic acid |
| 18 | 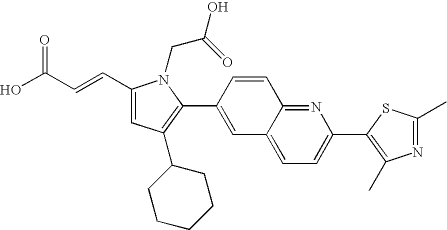 | (E)-3-(1-(carboxymethyl)-4-cyclohexyl-5-(2- (2,4-dimethylthiazol-5-yl)quinolin-6-yl)-1H- pyrrol-2-yl)acrylic acid |
| 19 | 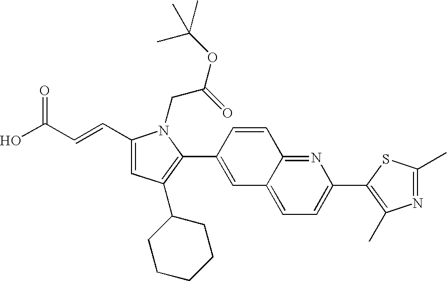 | (E)-3-(1-((tert-butoxycarbonyl)methyl)-4- cyclohexyl-5-(2-(2,4-dimethylthiazol-5- yl)quinolin-6-yl)-1H-pyrrol-2-yl)acrylic acid |
| 20 | 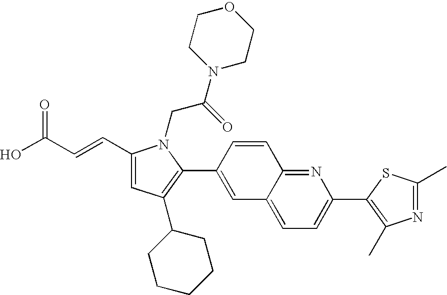 | (E)-3-(4-cyclohexyl-5-(2-(2,4-dimethylthiazol-5- yl)quinolin-6-yl)-1-(2-morpholino-2-oxoethyl)- 1H-pyrrol-2-yl)acrylic acid |
| 21 | 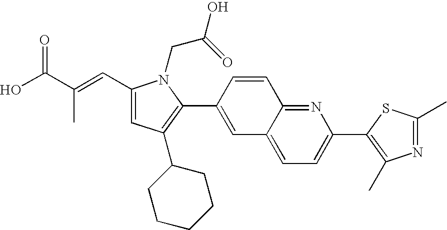 | (E)-3-(1-(carboxymethyl)-4-cyclohexyl-5-(2- (2,4-dimethylthiazol-5-yl)quinolin-6-yl)-1H- pyrrol-2-yl)-2-methylacrylic acid |
| 22 | 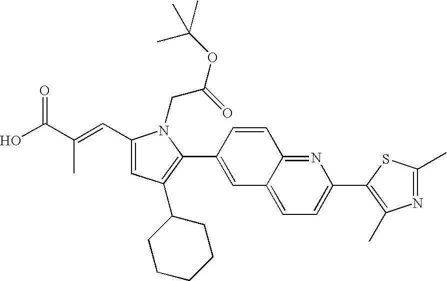 | (E)-3-(1-((tert-butoxycarbonyl)methyl)-4- cyclohexyl-5-(2-(2,4-dimethylthiazol-5- yl)quinolin-6-yl)-1H-pyrrol-2-yl)-2- methylacrylic acid |
| 23 | 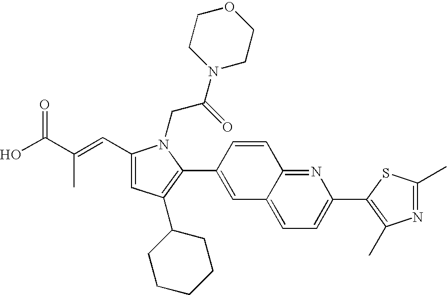 | (E)-3-(4-cyclohexyl-5-(2-(2,4-dimethylthiazol-5- yl)quinolin-6-yl)-1-(2-morpholino-2-oxoethyl)- 1H-pyrrol-2-yl)-2-methylacrylic acid |
| 24 | 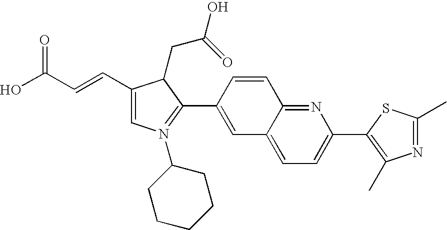 | (E)-3-(4-(carboxymethyl)-1-cyclohexyl-5-(2- (2,4-dimethylthiazol-5-yl)quinolin-6-yl)-1H- pyrrol-3-yl)acrylic acid |
| 25 | 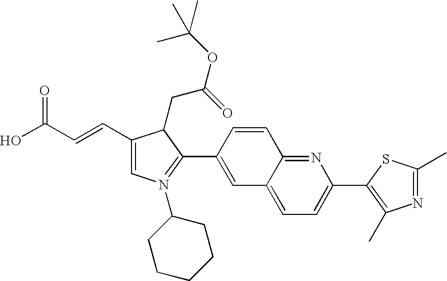 | (E)-3-(4-((tert-butoxycarbonyl)methyl)-1- cyclohexyl-5-(2-(2,4-dimethylthiazol-5- yl)quinolin-6-yl)-1H-pyrrol-3-yl)acrylic acid |
| 26 | 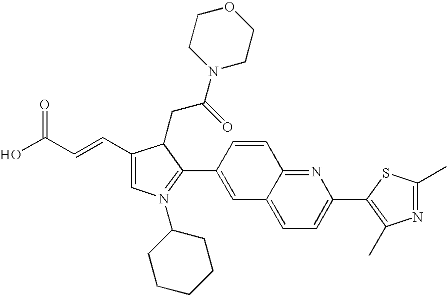 | (E)-3-(1-cyclohexyl-5-(2-(2,4-dimethylthiazol-5- yl)quinolin-6-yl)-4-(2-morpholino-2-oxoethyl)- 1H-pyrrol-3-yl)acrylic acid |
| 27 | 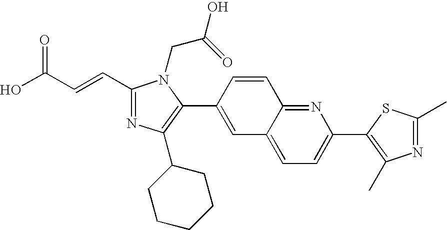 | (E)-3-(1-(carboxymethyl)-4-cyclohexyl-5-(2- (2,4-dimethylthiazol-5-yl)quinolin-6-yl)-1H- imidazol-2-yl)acrylic acid |
| 28 | 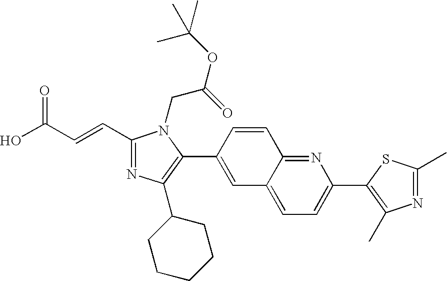 | (E)-3-(1-((tert-butoxycarbonyl)methyl)-4- cyclohexyl-5-(2-(2,4-dimethylthiazol-5- yl)quinolin-6-yl)-1H-imidazol-2-yl)acrylic acid |
| 29 | 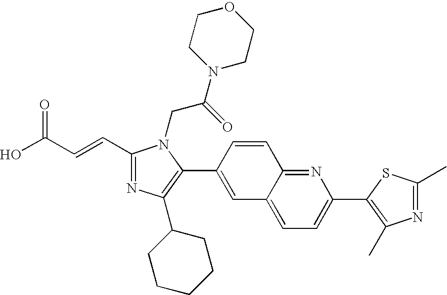 | (E)-3-(4-cyclohexyl-5-(2-(2,4-dimethylthiazol-5- yl)quinolin-6-yl)-1-(2-morpholino-2-oxoethyl)- 1H-imidazol-2-yl)acrylic acid |
| 30 | 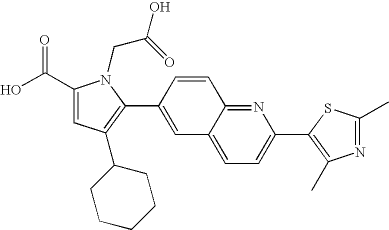 | 1-(carboxymethyl)-4-cyclohexyl-5-(2-(2,4- dimethylthiazol-5-yl)quinolin-6-yl)-1H-pyrrole- 2-carboxylic acid |
| 31 | 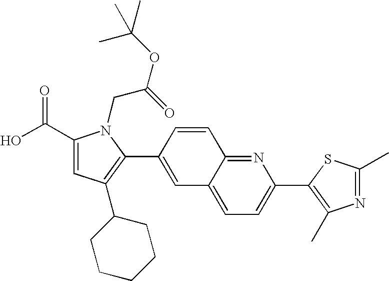 | 1-((tert-butoxycarbonyl)methyl)-4-cyclohexyl-5- (2-(2,4-dimethylthiazol-5-yl)quinolin-6-yl)-1H- pyrrole-2-carboxylic acid |
| 32 | 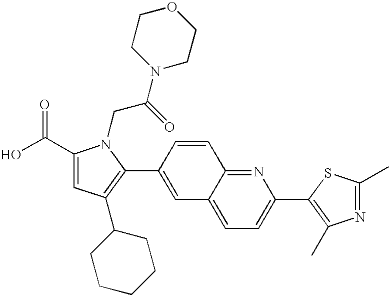 | 4-cyclohexyl-5-(2-(2,4-dimethylthiazol-5- yl)quinolin-6-yl)-1-(2-morpholino-2-oxoethyl)- 1H-pyrrole-2-carboxylic acid |
| 33 | 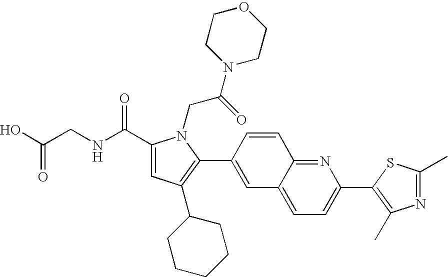 | 2-(4-cyclohexyl-5-(2-(2,4-dimethylthiazol-5- yl)quinolin-6-yl)-1-(2-morpholino-2-oxoethyl)- 1H-pyrrole-2-carboxamido)acetic acid |
| 34 | 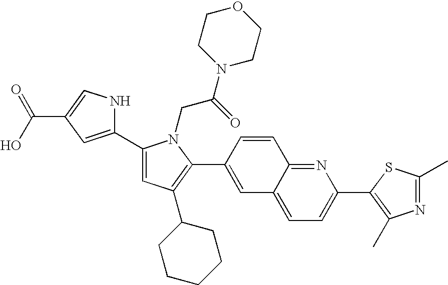 | 4′-cyclohexyl-5′-[2-(2,4-dimethyl-thiazol-5-yl)- quinolin-6-yl]-1′-(2-morpholin-4-yl-2-oxo- ethyl)-1H,1′H-[2,2′]bipyrrolyl-4-carboxylic acid |
| 35 | 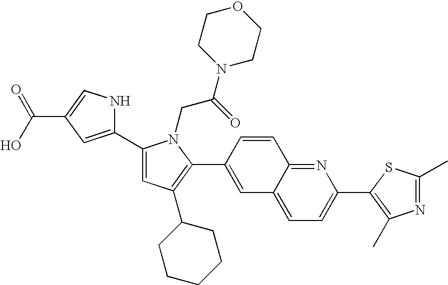 | 4-cyclohexyl-5-[2-(2,4-dimethyl-thiazol-5-yl)- quinolin-6-yl]-1-(2-morpholin-4-yl-2-oxo-ethyl)- 1H,1′H-[2,3′]bipyrrolyl-5′-carboxylic acid |
| 36 | 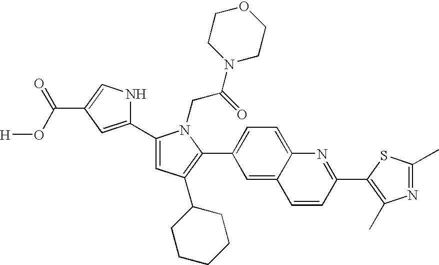 | 4′-cyclohexyl-5′-[2-(2,4-dimethyl-thiazol-5-yl)- quinolin-6-yl]-1′-(2-morpholin-4-yl-2-oxo- ethyl)-1H,1′H-[2,2′]bipyrrolyl-5-carboxylic acid |
| 37 | 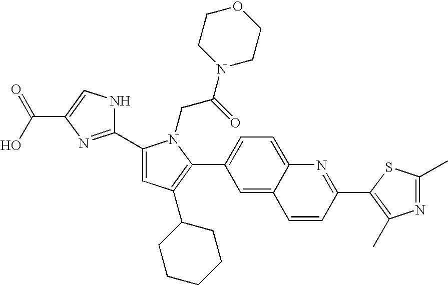 | 2-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-1H-imidazole-4- carboxylic acid |
| 38 | 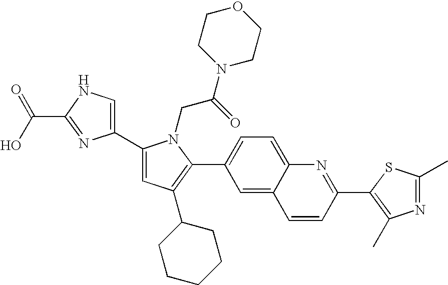 | 4-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-1H-imidazole-2- carboxylic acid |
| 39 | 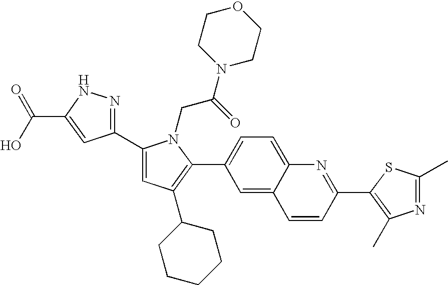 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-2H-pyrazole-3-carboxylic acid |
| 41 | 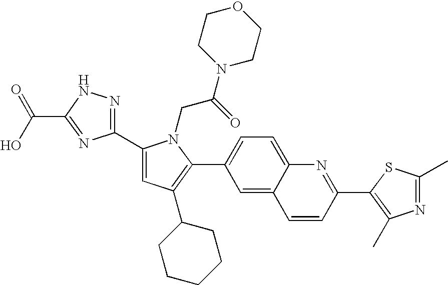 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-2H-[1,2,4]triazole-3- carboxylic acid |
| 42 | 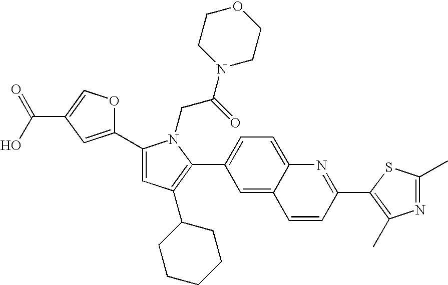 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-furan-3-carboxylic acid |
| 43 | 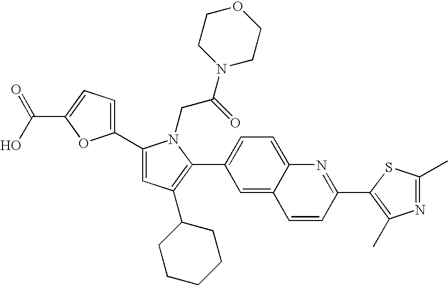 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-furan-2-carboxylic acid |
| 44 | 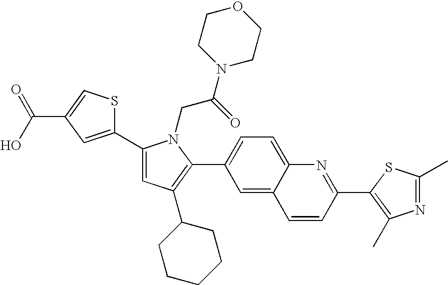 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-thiophene-3-carboxylic acid |
| 45 | 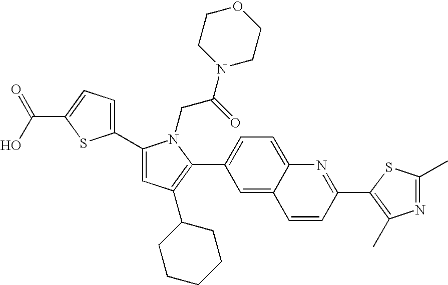 | 5-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-thiophene-2-carboxylic acid |
| 46 | 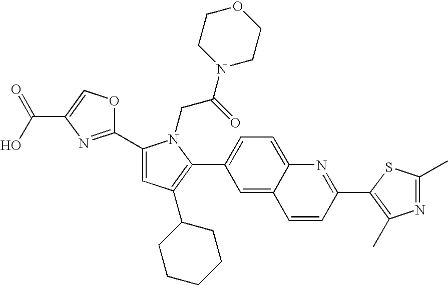 | 2-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-oxazole-4-carboxylic acid |
| 47 | 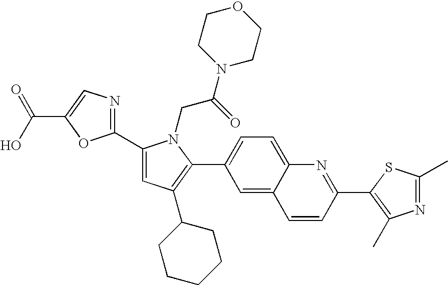 | 2-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-oxazole-5-carboxylic acid |
| 48 | 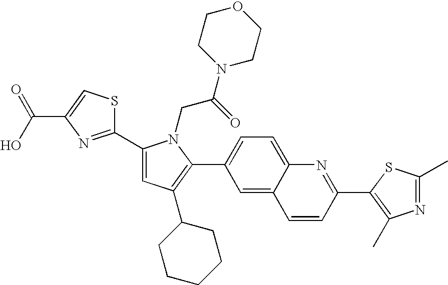 | 2-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-thiazole-4-carboxylic acid |
| 49 | 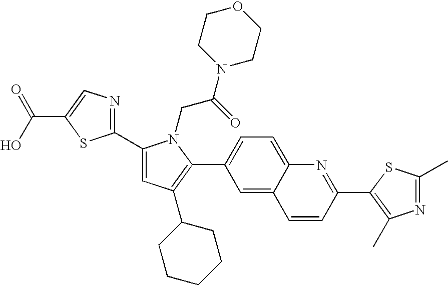 | 2-[4-cyclohexyl-5-[2-(2,4-dimethyl-thiazol-5- yl)-quinolin-6-yl]-1-(2-morpholin-4-yl-2-oxo- ethyl)-1H-pyrrol-2-yl]-thiazole-5-carboxylic acid |

Also provided are alkynyl compounds corresponding to compounds 1-20 and 24-29 wherein the alkenylene group L is replaced with an alkynylene group.

This invention is also directed to pharmaceutical compositions comprising a pharmaceutically acceptable diluent and a therapeutically effective amount of one of the compounds described herein or mixtures of one or more of such compounds.

This invention is further directed to uses of the compounds as described herein or mixtures of one or more of such compounds in the preparation of a medicament for treating a viral infection mediated, at least in part, by a virus in the Flaviviridae family of viruses, such as HCV.

This invention is still further directed to methods for treating a viral infection mediated at least in part by a virus in the flaviviridae family of viruses, such as HCV, in mammals which methods comprise administering to a mammal, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds.

In yet another embodiment of the invention, methods of treating or preventing viral infections in mammals are provided wherein the compounds of this invention are administered in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. Preferably, the additional agent active against HCV is interferon-alpha or pegylated interferon-alpha alone or in combination with ribavirin or viramidine.

Definitions

Unless otherwise indicated, this invention is not limited to any particular composition or pharmaceutical carrier, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “pharmaceutically acceptable diluent” in a composition includes two or more pharmaceutically acceptable diluents, and so forth.

In this specification and in the claims that follow, reference will be made to a number of terms that shall be defined to have the following meanings:

As used herein, “alkyl” refers to monovalent hydrocarbyl groups having from 1 to 10 carbon atoms, preferably from 1 to 5 carbon atoms, more preferably 1 to 3 carbon atoms, and also more preferably from 1 to 2 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic.

“Alkoxy” refers to the group “alkyl-O-” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O-”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Acylamino” refers to the group —C(O)NR^{f′}R^{g′} where R^{f′} and R^{g′} is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{f′} and R^{g′} are joined to form together with the nitrogen atom a heterocyclic or substituted heterocyclic ring.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

“Alkenyl” refers to hydrocarbyl groups having from 2 to 10 carbon atoms, preferably having from 2 to 6 carbon atoms, and more preferably 2 to 4 carbon atoms and having at least 1 and preferably from 1-2 sites of alkenyl unsaturation wherein each site of unsaturation independently has either cis or trans orientation or a mixture thereof.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to a vinyl carbon atom.

“Alkenylene” and “substituted alkenylene” refer to divalent alkenyl and substituted alkenyl groups as defined above. Preferred alkenylene and substituted alkenylene groups have two to three carbon atoms.

“Alkenyloxy” refers to the group alkenyl-O—.

“Alkylaryloxy” refers to the group alkyl-arylene-O—.

“Alkylthio” refers to the group alkyl-S—.

“Arylalkyloxy” refers to the group aryl-alkylene-O—.

“Alkynyl” refers to hydrocarbyl groups having from 2 to 10 carbon atoms, preferably having from 2 to 6 carbon atoms, and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to an acetylenic carbon atom.

“Alkynylene” and “substituted alkynylene” refer to divalent alkynyl and substituted alkynyl groups as defined above. Preferred alkynlene and substituted alkynylene groups have two to three carbon atoms.

“Alkylene” and “substituted alkylene” refer to divalent alkyl and substituted alkyl groups as defined above. Preferred alkylene and substituted alkylene groups have one to three or two to three carbon atoms.

“Amino” refers to the group —NH₂.

“Substituted amino” refers to the group-NR^{h′}R^{i′} where R^{h′} and R^{i′} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{h′} and R^{i′} are joined, together with the nitrogen bound thereto to form a heterocyclic or substituted heterocyclic group provided that R^{h′} and R^{i′} are both not hydrogen. When R^{h′} is hydrogen and R^{i′} is alkyl, the substituted amino group is sometimes referred to herein as alkylamino. When R^{h′} and R^{i′} are alkyl, the substituted amino group is sometimes referred to herein as dialkylamino.

“Aminoacyl” refers to the groups —NR^{j′}C(O)alkyl, —NR^{j′}C(O)substituted alkyl, —NR^{j′}C(O)-cycloalkyl, —NR^{j′}C(O)substituted cycloalkyl, —NR^{j′}C(O)alkenyl, —NR^{j′}C(O)substituted alkenyl, —NR^{j′}C(O)alkynyl, —NR^{j′}C(O)substituted alkynyl, —NR^{j′}C(O)aryl, —NR^{j′}C(O)substituted aryl, —NR^{j′}C(O)heteroaryl, —NR^{j′}C(O)substituted heteroaryl, —NR^{j′}C(O)heterocyclic, and —NR^{j′}C(O)substituted heterocyclic where R^{j′} is hydrogen or alkyl.

“Aminoalkyl” refers to the group amino-alkyl-.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl.

“Substituted aryl” refers to aryl groups which are substituted with from 1 to 3 substituents, and preferably 1 to 2 substituents, selected from the group consisting of hydroxy, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy esters, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Aralkyl” or “arylalkyl” refers to the group aryl-alkyl-.

“Arylene” and “substituted arylene” refer to divalent aryl and substituted aryl groups as defined above.

“Aryloxy” refers to the group aryl-O— that includes, by way of example, phenoxy, naphthoxy, and the like.

“Substituted aryloxy” refers to substituted aryl-O— groups.

“Carboxy” or “carboxyl” refers to —COOH or salts thereof.

“Carboxy esters” or “carboxyl esters” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic. Preferred carboxy esters are —C(O)O-alkyl, —C(O)O-substituted alkyl, —C(O)O-aryl, and —C(O)O-substituted aryl.

“Cycloalkyl” refers to cyclic alkyl groups of from 3 to 10 carbon atoms having single or multiple cyclic rings optionally comprising 1 to 3 exo carbonyl or thiocarbonyl groups. Suitable cycloalkyl groups include, by way of example, adamantyl, cyclopropyl, cyclobutyl, cyclopentyl, cyclohexyl, cyclooctyl, 3-oxocyclohexyl, and the like. In multiple condensed rings, one or more of the rings may be other than cycloalkyl (e.g., aryl, heteroaryl or heterocyclic) provided that the point of attachment is to a carbon ring atom of the cycloalkyl group.

“Substituted cycloalkyl” refers to a cycloalkyl group, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic. In one embodiment, the cycloalkyl group does not comprise 1 to 3 exo carbonyl or thiocarbonyl groups. In another embodiment, the cycloalkyl group does comprise 1 to 3 exo carbonyl or thiocarbonyl groups. It is understood, that the term “exo” refers to the attachment of a carbonyl or thiocarbonyl to a carbon ring atom of the cycloalkyl group. Substituted cyclopropyl is a species of substituted cycloalkyl and refers to a C₃ cycloalkyl substituted as above.

“Cycloalkenyl” refers to cyclic alkenyl but not aromatic groups of from 4 to 10 carbon atoms having single or multiple cyclic rings. Suitable cycloalkenyl groups include, by way of example, cyclopentyl, cyclohexenyl, and cyclooctenyl. In multiple condensed rings, one or more of the rings may be other than cycloalkenyl (e.g., aryl, heteroaryl or heterocyclic) provided that the point of attachment is to a carbon ring atom of the cycloalkyl group.

“Substituted cycloalkenyl” refers to cycloalkenyl groups, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic provided that for hydroxyl substituents the point of attachment is not to a vinyl carbon atom. Substituted cycloalkenyl also refers to cycloalkenyl groups optionally comprising 1 to 3 exo carbonyl or thiocarbonyl groups. It is understood, that the term “exo” refers to the attachment of a carbonyl or thiocarbonyl to a carbon ring atom of the cycloalkenyl group. Suitable 3-oxocyclohexenyl, and the like. In one embodiment, the cycloalkenyl group does not comprise 1 to 3 exo carbonyl or thiocarbonyl groups. In another embodiment, the cycloalkenyl group does comprise 1 to 3 exo carbonyl or thiocarbonyl groups.

“Cycloalkylene” and “substituted cycloalkylene” refer to divalent cycloalkyl and substituted cycloalkyl groups as defined above. Preferred cycloalkylene and substituted cycloalkylene groups have three to six carbon atoms.

“Cycloalkenylene” and “substituted cycloalkenylene” refer to divalent cycloalkenyl and substituted cycloalkenyl groups as defined above. Preferred cycloalkenylene and substituted cycloalkenylene groups have four to six carbon atoms.

“Cycloalkoxy” refers to —O-cycloalkyl groups.

“Substituted cycloalkoxy” refers to —O-substituted cycloalkyl groups.

The term “guanidino” refers to the group —NHC(═NH)NH₂ and the term “substituted guanidino” refers to —NR^{p′}C(═NR^{p′})N(R^{p′})₂ where each R^{p′} is independently hydrogen or alkyl.

“Halo” or “halogen” refers to fluoro, chloro, bromo and iodo and preferably is fluoro or chloro.

“Haloalkyl” refers to an alkyl group substituted with 1 to 10 halogen atoms.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur, within the ring. Preferably, such heteroaryl groups are aromatic groups of from 1 to 15 carbon atoms, preferably from 1 to 10 carbon atoms, and 1 to 4 heteroatoms selected from the group consisting of oxygen, nitrogen, and sulfur within the ring. Such heteroaryl groups can have a single ring (e.g., pyridyl or furyl) or multiple condensed rings (e.g., indolizinyl or benzothienyl). The sulfur atom(s) in the heteroaryl group may optionally be oxidized to sulfoxide and sulfone moieties.

“Substituted heteroaryl” refers to heteroaryl groups that are substituted with from 1 to 3 substituents selected from the same group of substituents defined for substituted aryl.

When a specific heteroaryl is defined as “substituted”, e.g., substituted qunioline, it is understood that such a heteroaryl contains the 1 to 3 substituents as recited above.

“Heteroarylene” and “substituted heteroarylene” refer to divalent heteroaryl and substituted heteroaryl groups as defined above.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated non-aromatic group having a single ring or multiple condensed rings, from 1 to 10 carbon atoms and from 1 to 4 hetero atoms selected from the group consisting of nitrogen, sulfur or oxygen within the ring which ring may optionally comprise 1 to 3 exo carbonyl or thiocarbonyl groups. Preferably, such heterocyclic groups are saturated or unsaturated group having a single ring or multiple condensed rings, from 1 to 10 carbon atoms and from 1 to 4 hetero atoms selected from the group consisting of nitrogen, sulfur, or oxygen within the ring. The sulfur atom(s) in the heteroaryl group may optionally be oxidized to sulfoxide and sulfone moieties.

In multiple condensed rings, one or more of the rings may be other than heterocyclic (e.g., aryl, heteroaryl or cycloalkyl) provided that the point of attachment is to a heterocyclic ring atom. In one embodiment, the heterocyclic group does not comprise 1 to 3 exo carbonyl or thiocarbonyl groups. In a preferred embodiment, the heterocyclic group does comprise 1 to 3 exo carbonyl or thiocarbonyl groups. It is understood, that the term “exo” refers to the attachment of a carbonyl or thiocarbonyl to a carbon ring atom of the heterocyclic group.

“Substituted heterocyclic” refers to heterocycle groups that are substituted with from 1 to 5 of the same substituents as defined for substituted cycloalkyl. Preferred substituents for substituted heterocyclic groups include heterocyclic groups having from 1 to 3 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic.

When a specific heterocyclic is defined as “substituted”, e.g., substituted morpholino, it is understood that such a heterocycle contains the 1 to 3 substituents as recited above.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“Hydroxy” or “hydroxyl” refers to —OH.

“Imino” refers to the group ═NR, where R is hydrogen, amino, alkyl, substituted alkyl, aryl, substituted aryl, or hydroxyl.

“Sulfonyl” refers to the group —SO₂—.

“Thiocarbonyl” refers to the group —C(═S)—.

“Thiol” refers to the group —SH.

“Thioalkyl” refers to the group HS-alkyl-.

The term “amino acid” refers to β-amino acids or to α-amino acids of the formula HR^{b′}N[CH(R^{a′})]_{c′}COOH where R^{a′} is an amino acid side chain, R^{b′} is hydrogen, alkyl, substituted alkyl or aryl and c′ is one or two. Preferably, c′ is one, an α-amino acid, and the α-amino acid is one of the twenty naturally occurring L amino acids.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR^{k′}, —PO₂(R^{k′})₂, —CN, —PO₃(R^{k′})₂, —OR^{k}, —SR^{k}, —NHCOR^{k′}, —N(R^{k′})₂, —CON(R^{k′)}₂, —CONH(O)R^{k′}, —CONHNHSO₂R^{k′}, —COHNSO₂R^{k′}, and —CONR^{k′}CN, where R^{k′} is selected from hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thiol, thioalkyl, alkylthio, sulfonyl, alkyl, alkenyl or alkynyl, aryl, aralkyl, cycloalkyl, heteroaryl, heterocycle, and CO₂R^{m′} where R^{m′} is hydrogen alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carboxylic acid isosteres contemplated by this invention.

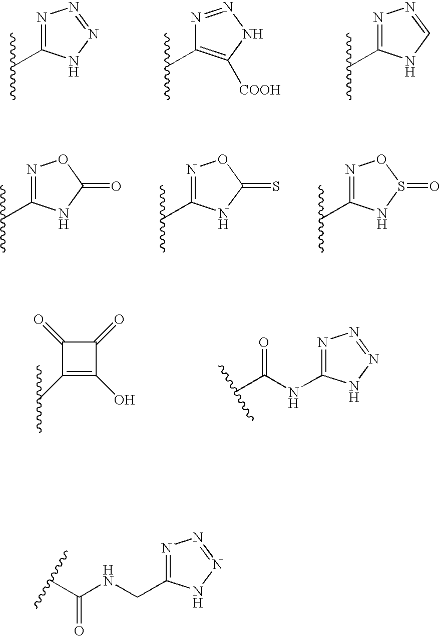

“Carboxylic acid bioisosteres” are compounds that behave as isosteres of carboxylic acids under biological conditions.

Other carboxylic acid isosteres not specifically exemplified or described in this specification are also contemplated by the present invention

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Prodrug” refers to any pharmaceutically acceptable salt, ester, salt of an ester, or other derivative of a compound of this invention that is capable of directly or indirectly providing a compound of this invention or an active metabolite or residue thereof when administered to a subject. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. An general overview of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

Similarly, it is understood that the above definitions are not intended to include impermissible substitution patterns (e.g., methyl substituted with 5 fluoro groups or a hydroxy group alpha to ethenylic or acetylenic unsaturation). Such impermissible substitution patterns are well known to the skilled artisan.

General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Compounds of the invention may generally be prepared in an analogous manner to that shown in Scheme 1 below. It is understood that for illustrative purposes, Scheme 1 employs the following substitution patterns: X is NR¹ where R¹ is methylenecarboxyl, methylene carboxylate or a 2-(2-morpholin-4-yl-2-oxoeth-1yl); Q is CH; X′ is C—R² where R² is cyclohexyl; L is a bond; Z is carboxyl, carboxylate or an amide derived from reaction with the amino group of an amino acid (e.g., glycine); Het is quinolin-2,6-ylene and Y is 2,4-dimethylthiazol-5-yl. Other compounds and substitution patterns can readily be made by the following the procedures below with proper substitution of reagents. Such factors are well within the skill of the art.

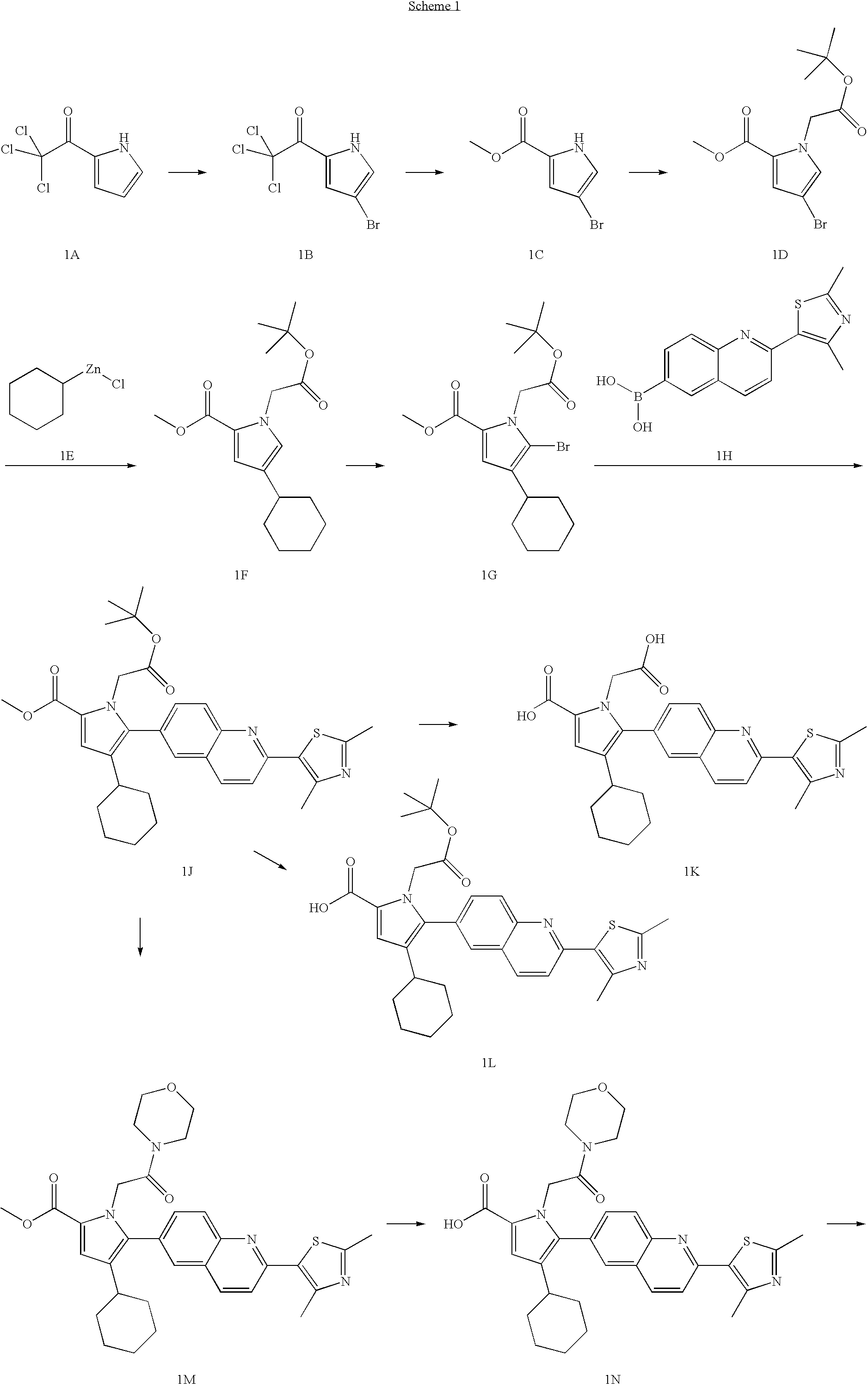

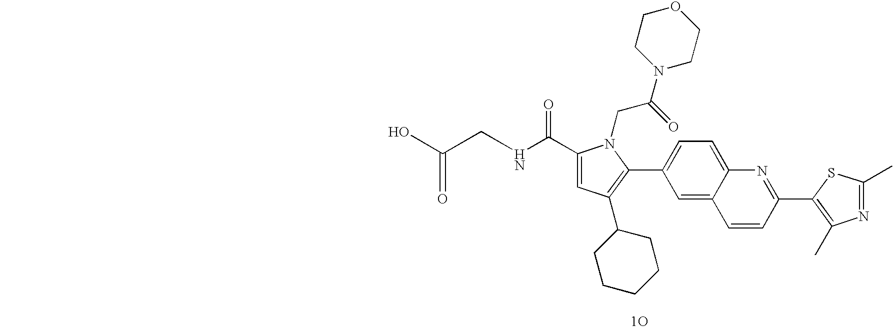

Specifically, in Scheme 1, commercially available 2,2,2-trichloro-1-(1H-pyrrol-2-yl)-ethanone, compound 1A (Aldrich, Milwaukee, Wis.), is contacted with an excess of bromine in the presence of a suitable inert diluent such as chloroform, carbon tetrachloride and the like. The reaction is typically conducted at a temperature of from −20° C. to about room temperature, although preferably around 0° C. The reaction is continued until it is substantially complete which typically occurs within about 0.2 to 10 hours. Upon completion of the reaction, compound 1B, 2,2,2-trichloro-1-(4-bromo-1H-pyrrol-2-yl)-ethanone, can be recovered by conventional methods including neutralization, evaporation, extraction, precipitation, chromatography, filtration, and the like or, alternatively, is employed in the next step without purification and/or isolation.

2,2,2-Trichloro-1-(4-bromo-1H-pyrrol-2-yl)-ethanone, compound 1B, is contacted with sodium methoxide to effect conversion to the methyl ester, compound 1C. This reaction proceeds by contacting compound 1B with an excess of sodium methoxide, typically from 1.1 to 5 equivalents and preferably 1.5 equivalents, in a suitable diluent such as methanol. The reaction is continued until it is substantially complete which typically occurs within about 1 to 30 minutes. Upon completion of the reaction, compound 1C, methyl 4-bromo-1H-pyrrole-2-carboxylate, can be recovered by conventional methods including neutralization, evaporation, extraction, precipitation, chromatography, filtration, and the like or, alternatively, is employed in the next step without purification and/or isolation.

Alkylation of the pyrrole amine of compound 1C proceeds via reaction with bromoacetic acid t-butyl ester. Specifically, compound 1C is contacted with an excess of a suitable base such sodium hydride in a suitable solvent such as DMF to facilitate the subsequent nucleophilic displacement reaction. Subsequently, a slight excess of an α-bromoacetic acid ester, e.g. t-butyl bromoacetate, is added to the reaction mixture and the reaction is maintained under ambient conditions until substantial completion which typically occurs within about 1 to 30 minutes. Upon completion of the reaction, compound 1D can be recovered by conventional methods including neutralization, evaporation, extraction, precipitation, chromatography, filtration, and the like or, alternatively, is employed in the next step without purification and/or isolation.

Introduction of the R² cyclohexyl group proceeds from compound 1D with in situ generated zincate 1E in the presence of Pd(P(tBu)₃)₂. In situ formation of the zincate preferably proceeds by contacting approximately equivalent amounts of cyclohexyl-magnesium chloride and zinc chloride in an inert solvent such as THF. The reaction is at ambient temperature for about 0.1 to 1 hours followed by addition of a higher boiling solvent such as NMP. To this mixture is added compound 1D and a slight excess of Pd(P(tBu)₃)₂. The reaction mixture is maintained under elevated temperature conditions, typically from about 80° to 120° C., until substantial completion which typically occurs within about 0.2 to 2 hours. Upon completion of the reaction, compound 1F can be recovered by conventional methods including neutralization, evaporation, extraction, precipitation, chromatography, filtration, and the like or, alternatively, is employed in the next step without purification and/or isolation.

Bromination of compound 1F proceeds under conventional conditions in the presence of pyridium tribromide to provide for compound 1G. Suzuki coupling of compound 1G with an excess of boronic acid 1H provides for compound 1J which can be recovered by conventional methods including neutralization, evaporation, extraction, precipitation, chromatography, filtration, and the like or, alternatively, is employed in the next step without purification and/or isolation.

Further functionalization of compound 1J using standard synthetic transformations provides for compounds 1K, 1L, and 1O. Specifically, conventional deesterification provides for compound 1K. Selective deprotection of the t-butyl ester followed by reaction with morpholine provides for compound 1M. Further deesterification of compound 1M provides for compound 1N. Conventional amino acid coupling to the carboxyl group of compound 1N using, e.g., glycine, provides for compound 1O.

A synthetic method for introducing an alkenylene linker is illustrated in Scheme 2. It is understood that for illustrative purposes, Scheme 2 employs the following substitution patterns: X is NR¹ where R¹ is 2-(2-morpholin-4-yl-2-oxoeth-1yl); Q is CH; X′ is C—R² where R² is cyclohexyl; L is vinyl (E isomer); Z is carboxyl; Het is quinolin-2,6-ylene and Y is 2,4-dimethylthiazol-5-yl. Other compounds and substitution patterns can readily be made by the following the procedures below with proper substitution of reagents. Such factors are well within the skill of the art.

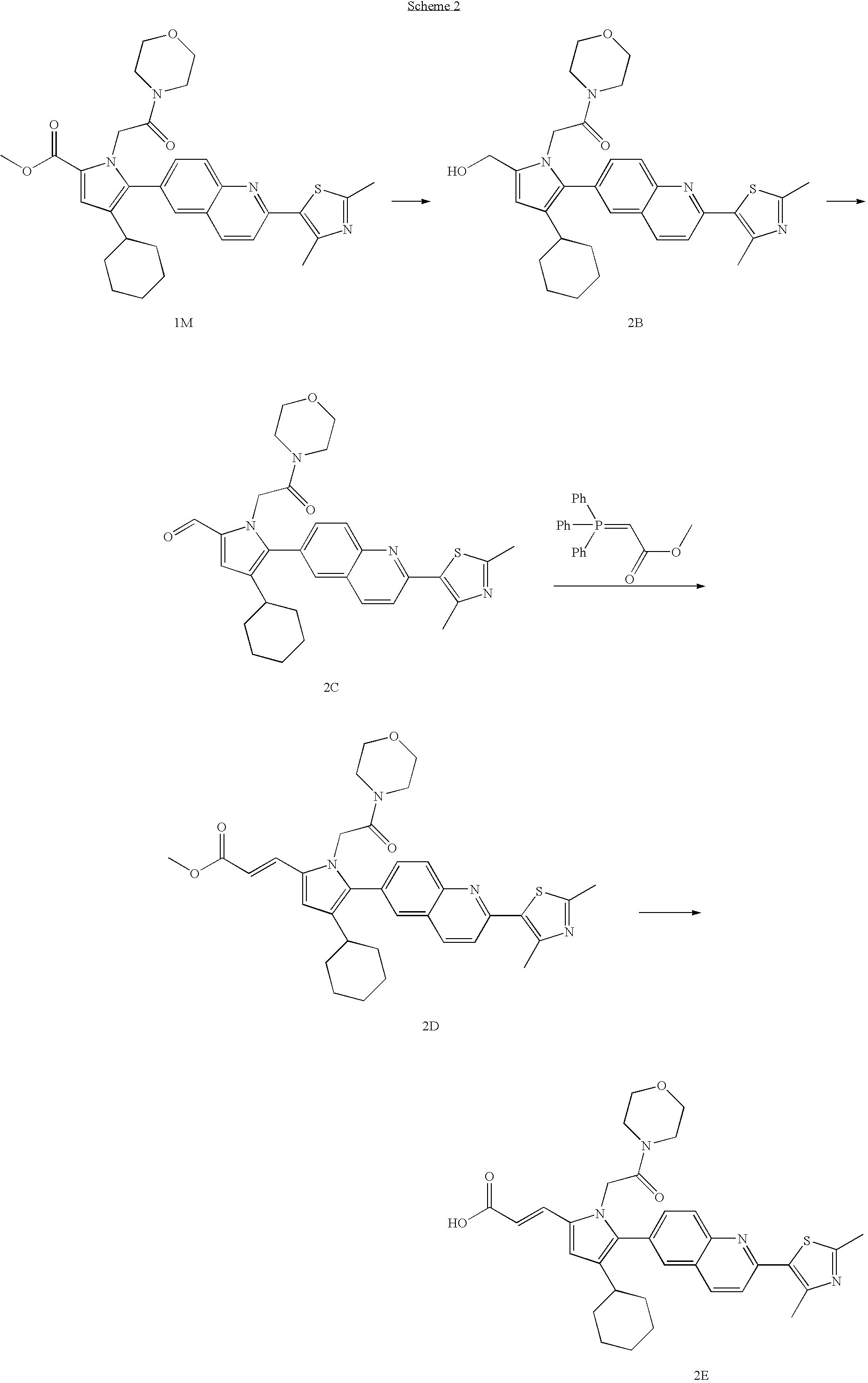

Specifically, in Scheme 2, compound 1M is reduced to the corresponding alcohol by a selective reducing agent (one which does not reduce the amide bond) such as lithium tri-t-butoxy aluminum hydride to provide for compound 2B. Subsequent oxidation to the aldehyde, compound 2C, proceeds via contact with a suitable oxidizing agent such as manganese dioxide. Wittig coupling using methyl (triphenylphosphoranyl-idene)acetate gives vinyl acetate 2D that can also be saponified to yield 2E.

Synthetic methods for modifying the alkenylene linkers are illustrated in Scheme 3. It is understood that for illustrative purposes, Scheme 3 employs the following substitution patterns: X is NR¹ where R¹ is 2-(2-morpholin-4-yl-2-oxoeth-1yl); Q is CH; X′ is C—R² where R² is cyclohexyl; Z is carboxyl; Het is quinolin-2,6-ylene and Y is 2,4-dimethylthiazol-5-yl. Other compounds and substitution patterns can readily be made by following the procedures below with proper substitution of reagents. Such factors are well within the skill of the art.

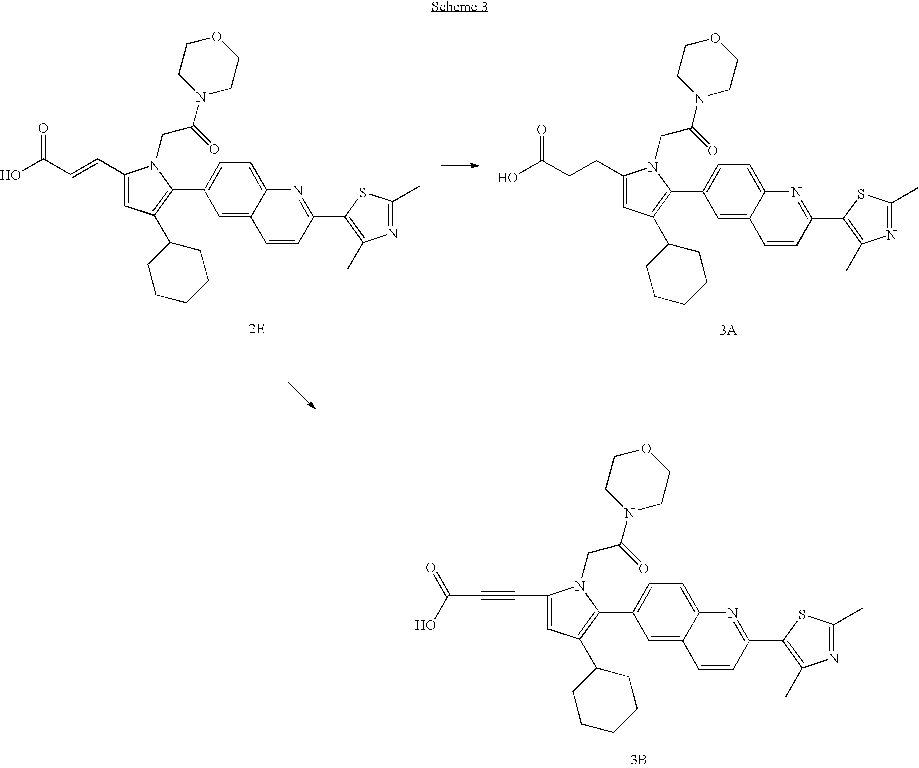

Specifically, in Scheme 3, the vinyl group of compound 2E (described above) is hydrogenated by conventional methods such as hydrogen over a palladium on carbon catalyst to provide for the ethylene linker of compound 3A. Alternatively, the vinyl group of compound 2E is 1,2 brominated under conventional conditions. Subsequent reaction with a suitable base such as potassium t-butoxide provides for compound 3B.

Synthetic methods for cyclizing the alkenylene linkers are illustrated in Scheme 4. It is understood that for illustrative purposes, Scheme 4 employs the following substitution patterns: X is NR¹ where R¹ is 2-(2-morpholin-4-yl-2-oxoeth-1yl); Q is CH; X′ is C—R² where R² is cyclohexyl; Z is carboxyl; Het is quinolin-2,6-ylene and Y is 2,4-dimethylthiazol-5-yl. Other compounds and substitution patterns can readily be made by following the procedures below with proper substitution of reagents. Such factors are well within the skill of the art.

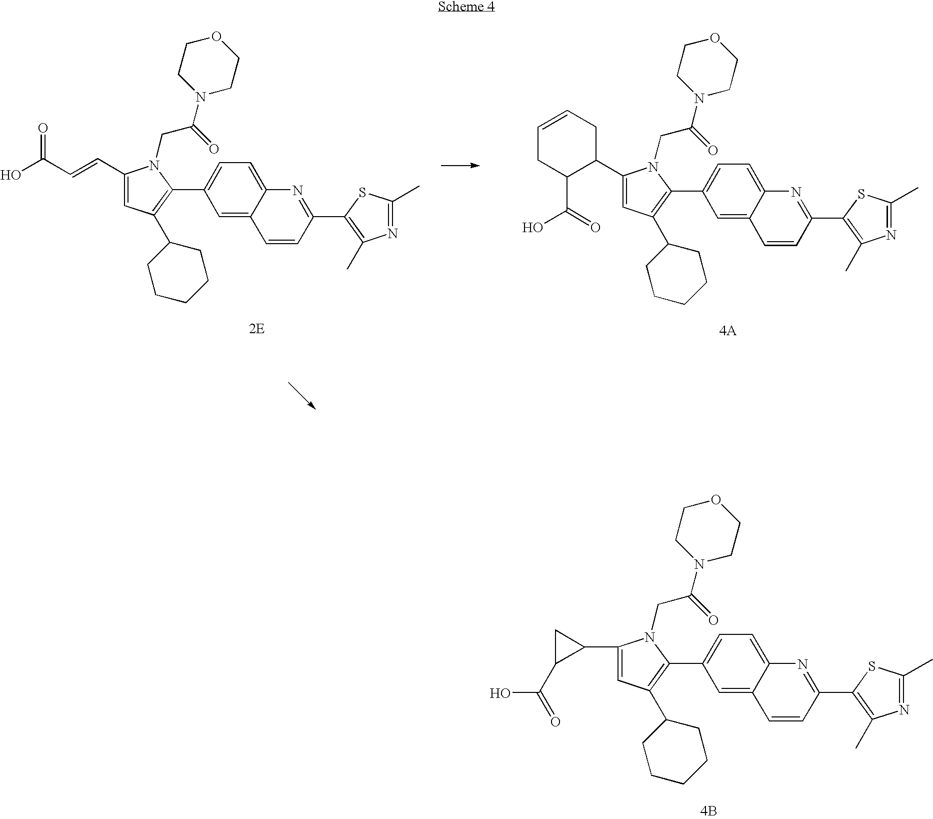

Specifically, the vinyl of compound 2E can be converted to the corresponding cyclopropyl group by conventional methods such as by reacting the vinyl group with a carbenoid to provide compound 4B. Alternatively, a Diels-Alder reaction on compound 2E would provide the cyclohexenyl derivative, compound 4A.

A method for introducing a heteroarylene linker is shown in Scheme 5. It is understood that for illustrative purposes, Scheme 5 employs the following substitution patterns: X is NR¹ where R¹ is 2-(2-morpholin-4-yl-2-oxoeth-1yl); Q is CH; X¹ is C—R² where R² is cyclohexyl; Z is carboxyl; Het is quinolin-2,6-ylene and Y is 2,4-dimethylthiazol-5-yl. Other compounds and substitution patterns can readily be made by following the procedures below with proper substitution of reagents. Such factors are well within the skill of the art.

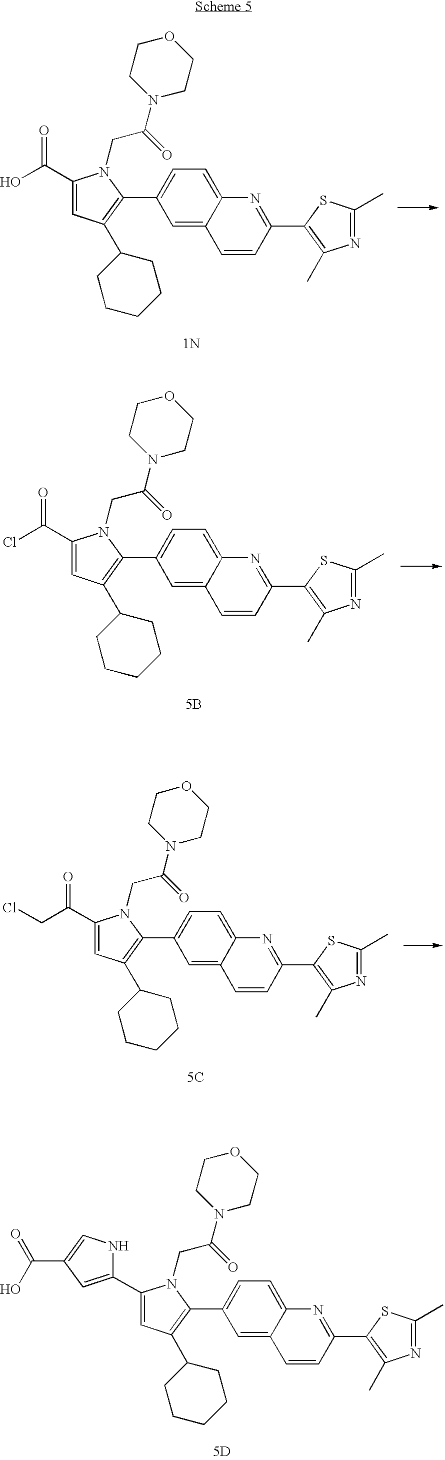

Specifically, in Scheme 5, acid 1N is converted to acid chloride 5B upon treatment with thionyl chloride. Reaction of 5B with less than two equivalents of diazomethane followed by treatment with HCl forms the chloromethyl ketone 5C. Compound 5C can be converted to acid 5D under Hantzsch pyrrole synthesis conditions. Accordingly, 5C is reacted with 3-oxo-propionic acid methyl ester CH₃OC(O)CH₂CHO in the presence of aqueous ammonia to form the methyl ester of 5D. Saponification of the ester with a base such as LiOH gives acid 5D.

Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

Compounds of this invention maybe used alone or in combination with other compounds to treat viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.1-25 mg/kg/day, more preferably from about 0.1 to 10 mg/kg/day. Thus, for administration to a 70 kg person, the dosage range would most preferably be about 7-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of formula 1 and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of formula 1 and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

### FORMULATION EXAMPLES

The following are representative pharmaceutical formulations containing a compound of formula I.

### Formulation Example 1

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| Ingredient | Quantity per capsule, mg |
|---|---|
| compound of this invention | 200 |
| lactose, spray-dried | 148 |
| magnesium stearate | 2 |

### Formulation Example 3

### Suspension Formulation

The following ingredients are mixed to form a suspension for oral administration. (q.s.=sufficient amount).

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4

### Injectable Formulation

The following ingredients are mixed to form an injectable formulation.

| Ingredient | Amount |
|---|---|
| compound of this invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| Compound of the invention | 500 mg |
| Witepsol ® H-15 | balance |

In the examples below and the synthetic schemes above, the following abbreviations have the following meanings. If an abbreviation is not defined, it has its generally accepted meaning.

| | |
|---|---|
| μL = | microliters |
| μM = | micromolar |
| μg = | micrograms |
| NMR = | nuclear magnetic resonance |
| boc = | t-butoxycarbonyl |
| br = | broad |
| d = | doublet |
| δ = | chemical shift |
| dd = | doublet of doublets |
| DIEA = | diisopropylethylamine |
| DMAP = | 4-N,N-dimethylaminopyridine |
| DMEM = | Dulbeco's Modified Eagle's Medium |
| DMF = | N,N-dimethylformamide |
| DMSO = | dimethylsulfoxide |
| DTT = | dithiothreotol |
| EDTA = | ethylenediaminetetraacetic acid |
| eq = | equivalent |
| ESI = | electrospray ionization |
| g = | gram |
| h or hr = | hours |
| HATU = | O-(7-Azabenzotriazol-1-yl)-N,N,N′,N′- |
| | tetramethyluronium hexafluorophosphate |
| HBTU = | O-Benzotriazol-1-yl-N,N,N′,N′- |
| | tetramethyluronium hexafluorophosphate |
| HCV = | hepatitus C virus |
| HPLC = | high performance liquid chromatography |
| Hz = | hertz |
| IPTG = | isopropyl-β-D-thiogalactopyranoside |
| IU = | International Units |
| IC₅₀ = | inhibitory concentration at 50% inhibition |
| J = | coupling constant (given in Hz unless |
| | otherwise indicated) |
| m = | multiplet |
| M = | molar |
| M + H⁺ = | parent mass spectrum peak plus H⁺ |
| mg = | milligram |
| mL = | milliliter |
| mM = | millimolar |
| mmol = | millimole |
| MS = | mass spectrum |
| nm = | nanometer |
| nM = | nanomolar |
| NMP = | 1-methyl-2-pyrrolidinone |
| ng = | nanogram |
| NTA = | nitrilotriacetic acid |
| NTP = | nucleoside triphosphate |
| PCR = | Polymerase chain reaction |
| ppm = | parts per million |
| psi = | pounds per square inch |
| Rp-HPLC = | reversed phase high performance liquid |
| | chromatography |
| s = | singlet |
| t = | triplet |
| TC₅₀ = | Toxic concentration at 50% cell toxicity |
| tetrakis or tetrakis | tetrakis(triphenylphosphine)palladium(0) |
| palladium = | |
| TFA = | trifluoroacetic acid |
| THF = | tetrahydrofuran |
| Tris = | Tris(hydroxymenthyl)aminomethane |
| UTP = | uridine triphosphate |

### Synthetic Examples

### Example 1

### Synthesis of 1-Carboxymethyl-4-cyclohexyl-5-[2-(2,4-dimethyl-thiazol-5-yl)-quinolin-6-yl]-1H-pyrrole-2-carboxylic acid (30)

### Step 1: Synthesis of 1-(4-Bromo-1H-pyrrol-2-yl)-2,2,2-trichloro-ethanone

2,2,2-Trichloro-1-(1H-pyrrol-2-yl)-ethanone (25 g, 117.7 mmol) was dissolved in 500 mL carbon-tetrachloride. Iodine (88 mg) was added and the mixture was cooled to 0 C°. A solution of 6.03 mL bromine in 50 mL carbon tetrachloride was added dropwise over a period of 30 minutes. The stirring was continued for an additional 30 minutes at the same temperature then the reaction mixture was transferred to a separatory funnel and was washed successively with 100 mL of 10% Na₂S₂O₃, saturated NaHCO₃ and brine (2×). It was then dried (sodium sulfate) and evaporated to dryness to give 33.9 g (98%) of 1-(4-bromo-1H-pyrrol-2-yl)-2,2,2-trichloro-ethanone as a white powder. H¹-NMR (DMSO-d₆): δ (ppm) 12.82 (s, 1H), 7.53 (m, 1H), 7.29 (m, 1H).

### Step 2: Synthesis of 4-Bromo-1H-pyrrole-2-carboxylic acid methyl ester

To a solution of 1-(4-bromo-1H-pyrrol-2-yl)-2,2,2-trichloro-ethanone, (28.9 g, 0.1 mol) in 500 mL methanol was added 25% NaOMe/MeOH (35 mL, 0.15 mol) dropwise. The reaction was complete in 10 minutes. The mixture was evaporated to dryness and solidified with icy water. The product was filtered off, washed with water until neutral, then dried to give 16.49 g (82%) of 4-bromo-1H-pyrrole-2-carboxylic acid methyl ester. MS: 203.96, 205.96 M+H⁺. H¹-NMR (DMSO-d₆): δ (ppm) 12.28 (s, 1H), 7.15 (m, 1H), 6.80 (m, 1H), 3.74 (s, 3H).

### Step 3: Synthesis of 4-Bromo-1-tert-butoxycarbonylmethyl-1H-pyrrole-2-carboxylic acid methyl ester

4-Bromo-1H-pyrrole-2-carboxylic acid methyl ester (4.9 mmol) was dissolved in DMF (5 mL), NaH (159 mg, 6.6 mmol) was added and the mixture was kept under vacuum for 15 minutes. Bromoacetic acid tert-butyl ester (760 μL, 5.15 mmol) was added in one portion and the solution was stirred for 5 minutes. The solvent was evaporated, the residue was taken up in a mixture of EtOAc and water, the organic phase was washed with water (1×), brine (2×), dried (MgSO₄) and evaporated to give 1.41 g (90%) of 4-bromo-1-tert-butoxycarbonylmethyl-1H-pyrrole-2-carboxylic acid methyl ester as a yellow oil which was pure enough to be used without further purification. MS: 339.9, 341.9 M+Na⁺. H¹-NMR (DMSO-d₆): δ (ppm) 7.23 (d, 1H, J=1.8 Hz), 6.83 (d, 1H, J=2.1 Hz), 4.88 (s, 2H), 3.63 (s. 3H), 1.34 (s, 9H).

### Step 4: Synthesis of 1-tert-Butoxycarbonylmethyl-4-cyclohexyl-1H-pyrrole-2-carboxylic acid methyl ester

To 22 mL 0.5M ZnCl₂ solution in THF was added 5.2 mL 2M cyclohexyl-magnesium chloride at room temperature. The mixture was stirred for 20 minutes then 15 mL NMP was added and the stirring was continued for 5 more minutes. 4-Bromo-1-tert-butoxycarbonylmethyl-1H-pyrrole-2-carboxylic acid methyl ester (1.095 g, 3.44 mmol) and 35 mg Pd(P(tBu)₃)₂ were then added. The mixture was heated at 100° C. for 40 minutes. The solvent was evaporated and the residue was purified on silica gel to yield 730 mg (66%) of 1-tert-butoxycarbonylmethyl-4-cyclohexyl-1H-pyrrole-2-carboxylic acid methyl ester. MS: 344.19 M+Na⁺. H¹-NMR (DMSO-d₆): δ (ppm) 6.89 (d, 1H, J=2.1 Hz), 6.71 (d, 1H, J=2.1 Hz), 4.86 (s, 2H), 3.65 (s, 3H), 2.37 (m, 1H), 1.86-1.61 (m, 3H), 1.40 s, 9H), 1.35-1.14 (m, 7H).

### Step 5: Synthesis of 5-Bromo-1-tert-butoxycarbonylmethyl-4-cyclohexyl-1H-pyrrole-2-carboxylic acid methyl ester

To an ice cold solution of 1-tert-butoxycarbonylmethyl-4-cyclohexyl-1H-pyrrole-2-carboxylic acid methyl ester (720 mg, 2.23 mmol) in 14 mL 1:1 THF-chloroform was added pyridinium tribromide (90%; 994 mg, 2.81 mmol) in one portion. The mixture was stirred under argon at the same temperature for 30 minutes and 3 mL 10% Na₂S₂O₃ solution was next added and the solution was stirred for 5 minutes. Chloroform (7 mL) was then added, and the organic phase was separated, washed with water (3×), sat. NaHCO₃ (1×), brine (2×), dried (Na₂SO₄), and evaporated. The product 5-bromo-1-tert-butoxycarbonylmethyl-4-cyclohexyl-1H-pyrrole-2-carboxylic acid methyl ester was a colorless oil, which later crystallized, in quantitative yield. MS: 422.0 and 424.0 M+Na⁺. H¹-NMR (DMSO-d₆): δ (ppm) 6.80 (s, 1H), 4.97 (s, 2H), 3.65 (s, 3H), 2.34 (m, 1H), 1.80-1.60 (m, 7H), 1.36 (s, 9H), 1.31-1.20 (m, 3H).

### Step 6: Synthesis of 1-tert-Butoxycarbonylmethyl-4-cyclohexyl-5-[2-(2,4-dimethyl-thiazol-5-yl)-quinolin-6-yl]-1H-pyrrole-2-carboxylic acid methyl ester

A mixture of 5-bromo-1-tert-butoxycarbonylmethyl-4-cyclohexyl-1H-pyrrole-2-carboxylic acid methyl ester (552 mg, 1.3 mmol), 2-(2,4-dimethyl-thiazol-5-yl)-quinoline-6-boronic acid (522 mg, 1.83 mmol; below), tetrakis(triphenylphosphino)-palladium(0) (78 mg, 0.07 mmol), 26 mL DMF, 26 mL methanol, and 3.1 mL saturated NaHCO₃ was heated at 80° C. for 1 h and then evaporated to dryness and purified on silica gel using hexane-ethyl acetate eluent system. Yield: 564 mg (77%) 1-tert-butoxycarbonylmethyl-4-cyclohexyl-5-[2-(2,4-dimethyl-thiazol-5-yl)-quinolin-6-yl]-1H-pyrrole-2-carboxylic acid methyl ester as a yellow oil. MS: 560.25 M+H⁺. H¹-NMR (DMSO-d₆): δ (ppm) 8.44 (d, 1H, J=9 Hz), 8.02 (d, 1H, J=8.7 Hz), 7.90-7.87 (m, 2H), 7.58 (dd, 1H, J=8.4 Hz), 6.93 (s, 1H), 4.70 (s, br, 2H), 3.73 (s, 3H), 2.7 (s, 3H), 2.66 (s, 3H), 2.29 (m, 1H), 1.70-1.11 (m, 19H).

### Synthesis of 2-(2,4-dimethyl-thiazol-5-yl)-quinoline-6-boronic acid

A mixture of 2-amino-5-bromobenzaldehyde (1.071 g, 5.354 mmol), 5-acetyl-2,4-dimethylthiazole (723 μL, 5.354 mmol) and 9.0 mL 10% KOH/ethanol (16.062 mmol KOH) in 60 mL ethanol was refluxed overnight under argon. It was then evaporated and the residue triturated with water. The solid crude product was filtered through a 250 mL silica pad using a 10% to 60% toluene-ethylacetate gradient to give 1.164 g (68%) of 6-bromo-2-(2,4-dimethylthiazol-5-yl)quinoline: ¹H-NMR (DMSO-d₆): δ (ppm) 8.39 (d, 1H, J=8.7 Hz), 8.27 (m, 1H), 7.88-7.86 (m, 3H), 2.68 (s, 3H), 2.64 (s, 3H). A DMSO solution of the product bromide, potassium acetate (3 eq.), P(Ph)₃Pd(II)Cl₂ catalyst (0.05 eq.) and bis(neopentylglycolato)diboron (3 eq.) was heated at 50° C. under argon for 4 h. After 150 mL water and 150 mL ethyl acetate was added, the organic phase was separated. The aqueous phase was extracted one more time with 50 mL ethyl acetate. The organic phases were pooled and washed with water (2×), brine (2×) and dried (sodium sulfate). The solvent was evaporated and the residue was purified by filtering through a 400 mL silica pad using toluene-ethyl acetate gradient to get 4.4 g (84%) of the title compound

MS: 285.08 (M+H⁺);

¹H-NMR (DMSO-d₆): δ (ppm) 8.47 (d, 1H, J=8.7 Hz), 8.33 (s, 1H), 7.97 (m, 1H), 7.88-7.79 (m, 2H), 2.69 (s, 3H), 2.64 (s, 3H).

### Step 7: Synthesis of 1-Carboxymethyl-4-cyclohexyl-5-[2-(2,4-dimethyl-thiazol-5-yl)-quinolin-6-yl]-1H-pyrrole-2-carboxylic acid

To a solution of 1-tert-butoxycarbonylmethyl-4-cyclohexyl-5-[2-(2,4-dimethyl-thiazol-5-yl)-quinolin-6-yl]-1H-pyrrole-2-carboxylic acid methyl ester 140 mg (0.25 mmol) in 5 mL dioxane and 1 mL methanol was added 3 mL of 2M NaOH and the mixture was heated at 55° C. for 2 h. The solvent was removed by evaporation and residue was purified by RP-HPLC to give 41 mg (30%) of 1-carboxymethyl-4-cyclohexyl-5-[2-(2,4-dimethyl-thiazol-5-yl)-quinolin-6-yl]-1H-pyrrole-2-carboxylic acid. MS: 490.1 M+H⁺. H¹-NMR (DMSO-d₆): δ (ppm) 8.5 (d, 1H, J=8.7 Hz), 8.03 (d, 1H, J=8.7 Hz) 7.91-7.88 (m, 2H), 7.60 (dd, 1H, J=8.4 & 1.8 Hz), 6.87 (s, 1H), 4.74 (s, br, 2H), 2.72 (s, 3H), 2.70 (s, 3H), 2.28 (m, 1H), 1.70-1.05 (m, 10H).

### Example 2

### Synthesis of 1-tert-Butoxycarbonylmethyl-4-cyclohexyl-5-[2-(2,4-dimethyl-thiazol-5-yl)-quinolin-6-yl]-1H-pyrrole-2-carboxylic acid (31)

To a solution of 1-tert-butoxycarbonylmethyl-4-cyclohexyl-5-[2-(2,4-dimethyl-thiazol-5-yl)-quinolin-6-yl]-1H-pyrrole-2-carboxylic acid methyl ester (50 mg, 0.09 mmol) in methanol-dioxane 1:1, was added 447 μL 1M NaOH and the mixture was stirred at 40° C. for 1 h when it was evaporated and purified by RP-HPLC to give 5.1 mg (10%) of 1-tert-Butoxycarbonylmethyl-4-cyclohexyl-5-[2-(2,4-dimethyl-thiazol-5-yl)-quinolin-6-yl]-1H-pyrrole-2-carboxylic acid. MS: 546.1 M+H⁺. H¹-NMR (DMSO-d₆): δ (ppm) 8.43 (d, 1H, J=9 Hz), 8.02 (d, 1H, J=9 Hz), 7.9-7.87 (m, 2H), 7.58 (dd, 1H, J=8.7 & 1.8 Hz), 6.88 (s, 1H), 4.7 (s, br, 2H), 2.71 (s, 3H), 2.66 (s, 3H), 2,28 (m, 1H), 1.7-1.11 (m, 19H).

### Example 3

### Synthesis of 4-Cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid (32)

### Step 1: Synthesis of 4-Cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid methyl ester

1-tert-Butoxycarbonylmethyl-4-cyclohexyl-5-[2-(2,4-dimethyl-thiazol-5-yl)-quinolin-6-yl]-1H-pyrrole-2-carboxylic acid methyl ester (514 mg, 0.92 mmol) was treated with a mixture of 20 mL TFA and 4 mL anisole at room temperature for 1 h. The reagents were evaporated to dryness to give 722 mg yellow oil. 620 mg of this oil was coupled with 88 μL morpholine by means of 859 mg HBTU and 875 μL DIEA in DMF (12 mL) using general preactivation procedure. When the reaction was complete (10 minutes) the DMF was evaporated, the residue was taken up in ethyl acetate, washed successively with water, dilute HCl, water, sodium bicarbonate solution and brine then was dried (sodium sulfate) and evaporated to yield 527 mg of 4-cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid methyl ester as a yellow oil which was pure enough to be used in the next step. MS: 573.25 M+H⁺. H¹-NMR (DMSO-d₆): δ (ppm) 8.46 (d, 1H, J=8.4 Hz), 8.01 (d, 1H, J=8.7 Hz), 7.89-7.86 (m, 2H), 7.59 (dd, 1H, J=8.7 & 1.8 Hz), 6.91 (s, 1H), 4.92 (s, 2H), 3.71 (s, 3H), 3.49-3.38 (m, 8H), 2.69 (s, 3H), 2.66 (s, 3H), 2.30 (m, 1H), 1.71-1.10 (m, 10H).

### Step 2: Synthesis of 4-Cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid

The oil 4-cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid methyl ester was dissolved in 10 mL methanol and 3 mL 1M NaOH was added and the solution was stirred for 4 h when the solvent was evaporated. The residue was purified by RP-HPLC to give 30.2 mg of 4-cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid as a yellow solid. MS: 559.1 M+H⁺. H¹-NMR (DMSO-d₆): δ (ppm) 8.47 (d, 1H J=8.7 Hz), 8.02 (d, 1H, 9 Hz), 7.90-7.87 (m, 2H), 7.60 (dd, 1H, J=8.7 & 1.8 Hz), 6.85 (s, 1H), 4.92 (s, 2H), 3.47-3.33 (m, 8H), 2.71 (s, 3H), 2.68 (s, 3H0, 2.29 (m, 1H), 1.75-1.06 (m, 10H).

### Example 4

### Synthesis of {[4-Cyclohexyl-5-[2-(2,4-dimethyl-thiazol-5-yl)-quinolin-6-yl]-1-(2-morpholin-4-yl-2-oxo-ethyl)-1H-pyrrole-2-carbonyl]-amino}-acetic acid (33)

4-Cyclohexyl-5-[2-(2,4-dimethyl-thiazol-5-yl)-quinolin-6-yl]-1-(2-morpholin-4-yl-2-oxo-ethyl)-1H-pyrrole-2-carboxylic acid (80 mg, 0.143 mmol) was coupled with glycine-methyl ester (27 mg, 0.215 mmol) using HBTU/DIEA. The methyl ester was then saponified in a mixture of 5 mL THF, 4 mL methanol and 1 mL 1M NaOH at room temperature for 30 minutes when it was evaporated and purified with RP-HPLC. Yield: 29.6 mg (34%) of {[4-cyclohexyl-5-[2-(2,4-dimethyl-thiazol-5-yl)-quinolin-6-yl]-1-(2-morpholin-4-yl-2-oxo-ethyl)-1H-pyrrole-2-carbonyl]-amino}-acetic acid as yellow solid. MS: 616.25 M+H⁺. H¹-NMR (DMSO-d₆): δ (ppm) 8.50 (d, 1H), 8.0 (d, 1H), 7.9-7.85 (m, 2H), 7.60 (dd, 1H), 6.95 (s, 1H), 5.00 (s, 2H), 3.82 (d, 2H), 3.37-3.29 (m, 8H), 2.71 (s, 3H), 2.68 (s, 3H), 2.31 (m, 1H), 1.75-1.05 (m, 10H).

### Biological Examples

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture is disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application Ser. No. 60/004,383, filed on September 1995, describes an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1(Supp 4) 18-24.

Screens that measure reductions in kinase activity from HCV drugs are disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs are disclosed in U.S. Pat. No. 5,861,267 to Su et al., U.S. Pat. No. 5,739,002 to De Francesco et al., and U.S. Pat. No. 5,597,691 to Houghton et al.

### Example 2

### Replicon Assay

A cell line, ET (Huh-lucubineo-ET) was used for screening of compounds of the present invention for HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding nucleoside analogs. Then the compounds were added to the cells to achieve a final concentration of 5 or 50 μM. Luciferase activity was measured 48-72 hours later by adding a lysis buffer and the substrate (Catalog number Glo-lysis buffer E2661 and Bright-Glo leuciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing potent antiviral activities, but no significant cytotoxicities were chosen for further evaluation. For these determinations, a 10-point, 2-fold serial dilution for each compound was used which spans a concentration range of 1000 fold. IC₅₀ and TC₅₀ values were calculated by fitting % inhibition at each concentration to the following equation:
% inhibition=100%/[(IC50/[I])^{b}+1]
where b is Hill's coefficient.

The % inhibition at a particular concentration was determined using the following equation:
% Inhibition=100−[100*(Lum with inhibitor-bg)/(Lum with no inhibitor-bg)]
where bg was the background with no replicon cell, and Lum was the luminescence intensity of the reporter luciferase gene.

In this assay, when tested at 33 μM, compounds 30, 31, 32 and 33 exhibited 22%, 48%, 57% and 17% inhibitions, respectively.

### Example 3

### Cloning and Expression of Recombinant HCV-NS5b

The coding sequence of NS5b protein is cloned by PCR from pFKI₃₈₉luc/NS3-3′/ET as described by Lohmann, V., et al. (1999) Science 285, 110-113 using the primers shown on page 266 of WO 2005/012288

The cloned fragment is missing the C terminus 21 amino acid residues. The cloned fragment is inserted into an IPTG-inducible expression plasmid that provides an epitope tag (His)6 at the carboxy terminus of the protein.

The recombinant enzyme is expressed in XL-1 cells and after induction of expression, the protein is purified using affinity chromatography on a nickel-NTA column. Storage condition is 10 mM Tris-HCl pH 7.5, 50 mM NaCl, 0.1 mM EDTA, 1 mM DTT, 20% glycerol at −20° C.

### Example 4

### HCV-NS5b Enzyme Assay

The polymerase activity is assayed by measuring incorporation of radiolabeled UTP into a RNA product using a biotinylated, heteropolymeric template, which includes a portion of the HCV genome. Typically, the assay mixture (50 μL) contains 10 mM Tris-HCl (pH 7.5), 5 mM MgCl₂, 0.2 mM EDTA, 10 mM KCl, 1 unit/μL RNAsin, 1 mM DTT, 10 μM each of NTP, including [³H]-UTP, and 10 ng/μL heteropolymeric template. Test compounds are initially dissolved in 100% DMSO and further diluted in aqueous buffer containing 5% DMSO. Typically, compounds are tested at concentrations between 1 nM and 100 μM. Reactions are started with addition of enzyme and allowed to continue at 37° C. for 2 hours. Reactions are quenched with 8 μL of 100 mM EDTA and reaction mixtures (30 μL) are transferred to streptavidin-coated scintillation proximity microtiter plates (FlashPlates) and incubated at 4° C. overnight. Incorporation of radioactivity is determined by scintillation counting.

What is claimed is:

1. A compound of the formula (I): 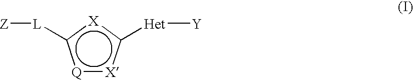
wherein:
L is selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;
one of X or X′ is N—R¹ and the other is selected from the group consisting of C—R², N, O or S;
Q is selected from the group consisting of C—R, N, O or S with the proviso that when X or X′ is O or S, then Q is selected from C—R and N;
R is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl;
R¹ and R² are independently selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CH₂CONR³R⁴, and —NR³R⁴; where each of R^{1a}, R³ and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl;
Z is selected from the group consisting of:
(a) hydrogen, halo, alkyl, substituted alkyl, alkenyl, substituted alkenyl, alkoxy, substituted alkoxy, cyano, aryl, substituted aryl, heteroaryl, substituted heteroaryl, amino, and substituted amino;
(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;
(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic, and substituted heterocyclic or, alternatively, R⁵ and R⁶ together with the nitrogen atom pendent thereto, form a heterocyclic, a substituted heterocyclic, a heteroaryl or a substituted heteroaryl ring group;
(d) —(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each R⁷, R¹⁰ and R¹¹ is independently hydrogen, alkyl, substituted alkyl, cycloalkyl, or substituted cycloalkyl, and wherein each R⁷ and R¹⁰ is optionally substituted with at least one halo, hydroxy, carboxy, carboxy ester, alkyl, alkoxy, amino, substituted amino; or alternatively, R⁷ and R¹⁰ or R¹⁰ and R¹¹ together with the atoms bound thereto join together to form an optionally substituted heterocyclic group;
(e) —C(X³)—N(R¹²)CR¹³R^{13′}C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above;
R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or, alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;
R¹² is selected from hydrogen and alkyl or, when R¹³ and R^{13′} are not taken together to form a ring and when R¹³ or R^{13′} and R¹⁰ or R¹¹ are not joined to form a heterocyclic or substituted heterocyclic group, then R¹², together with the nitrogen atom pendent thereto, may be taken together with one of R¹³ and R^{13′} to form a heterocyclic or substituted heterocyclic ring group;
(f) —C(X²)—N(R¹²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and
(g) carboxylic acid isostere;
with the proviso that when L is a bond, Z is not hydrogen;
Het is selected from the group consisting of arylene, substituted arylene, heteroarylene and substituted heteroarylene; and
Y is selected from the group consisting of alkyl, aryl, heteroaryl, substituted aryl, and substituted heteroaryl;
or a pharmaceutically acceptable salt, ester, stereoisomer, prodrug, or tautomer thereof.

2. A compound of claim 1 having the formula (II), (III), or (IV): 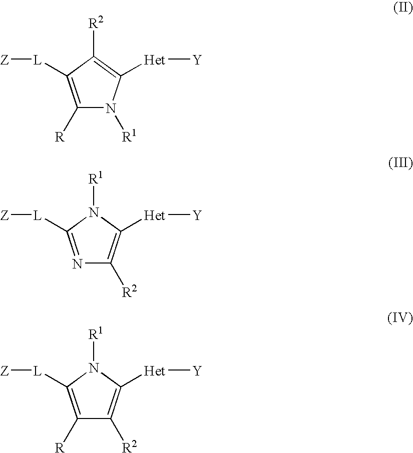
wherein Z, L, R, R¹, R², Het, and Y are previously defined.

3. A compound of claim 1 wherein R is hydrogen, halo, or methyl.

4. A compound of claim 3 wherein R is hydrogen.

5. A compound of claim 1 wherein Z is —COOH, —COOR^{z}, 1H-tetrazol-5-yl, —C(O)NHSO₂CF₃,

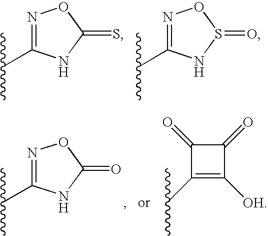

6. A compound of claim 5 wherein Z is —COOH.

7. A compound of claim 6 wherein L is a bond.

8. A compound of claim 6 wherein L is —CH═CH— or —(CH₃)C═CH—, each having either a cis or trans orientation.

9. A compound of claim 1 wherein Het is heteroarylene or substituted heteroarylene, Y is aryl, heteroaryl, substituted aryl, or substituted heteroaryl, and Het and Y together form a -Het-Y group.

10. A compound of claim 9 wherein said -Het-Y group has the formula (H1) 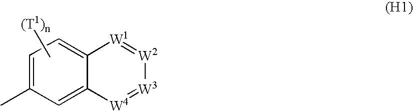
wherein each W¹, W², W³ and W⁴ are independently selected from N, CH, CT², and C—Y; provided that no more than 2 of W¹, W², W³ and W⁴ are N; provided that one of W¹, W², W³ and W⁴ is C—Y; and further provided wherein no more than one N in the ring system is optionally oxidized to form the N-oxide;
T¹ and T² are independently selected from the group consisting of alkyl, substituted alkyl, alkoxy, substituted alkoxy, amino, substituted amino, cyano, carboxyl, carboxyl ester, halo, hydroxy, heterocyclic, substituted hetereocyclic, and nitro; and
n is an integer equal to 0, 1, or 2.

11. A compound of claim 10 wherein said -Het-Y group has the formula (H2) 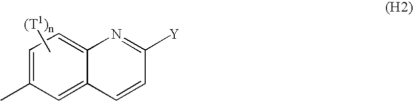
wherein T¹, n, and Y are previously defined.

12. A compound of claim 1 wherein said -Het-Y group is

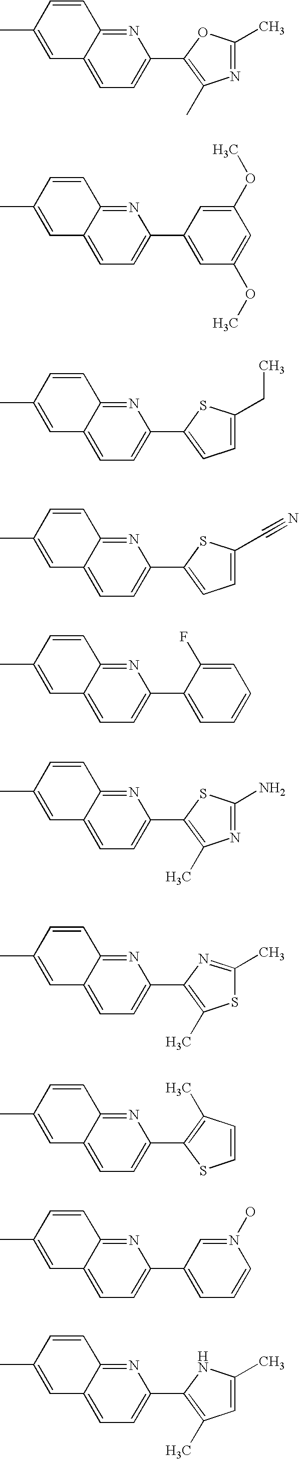

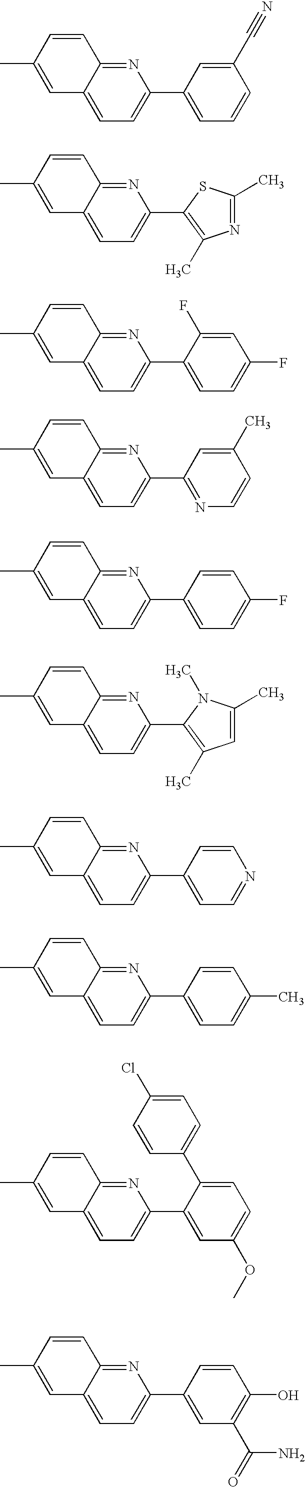

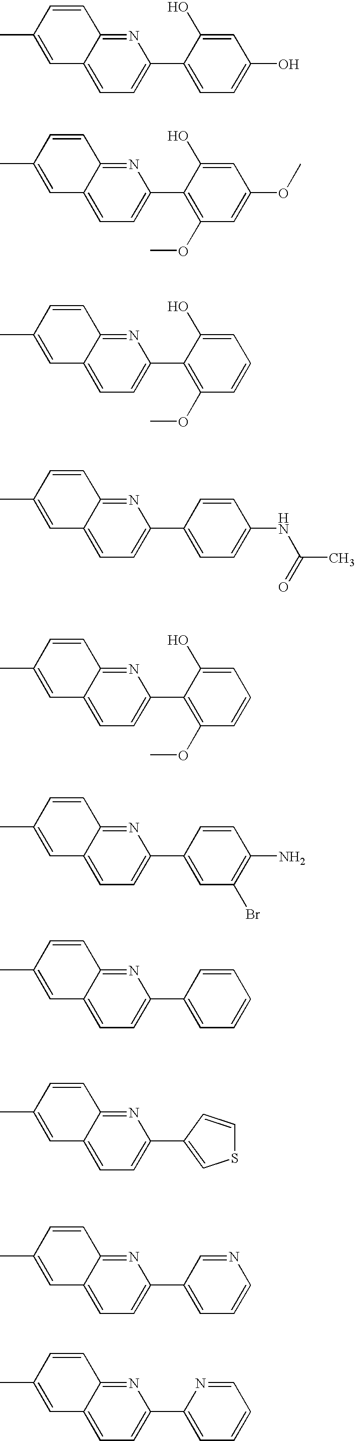

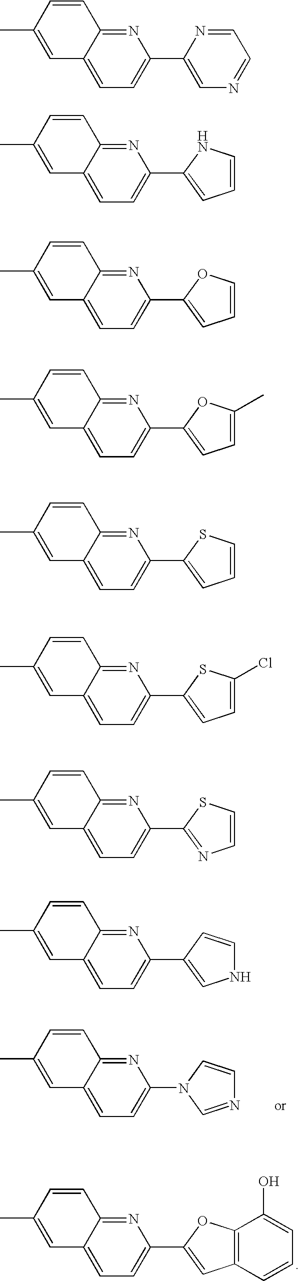

13. A compound of claim 1 wherein R¹ or R² is selected from the group consisting of —COOH, —CH₂COOR^{1a}, and —CH₂CONR³R⁴ when said R¹ or R² is attached to a ring atom adjacent to a ring atom bearing L.

14. A compound of claim 1 wherein R¹ or R² is cyclohexyl when said R¹ or R² is attached to a ring atom adjacent to a ring atom bearing R.

15. A compound of claim 1 having the formula (V): 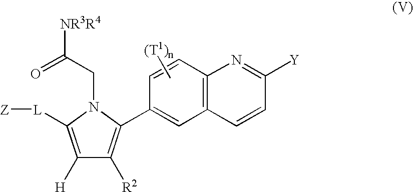
wherein Z, L, R², R³, R⁴, and Y are previously defined;
T¹ is selected from the group consisting of alkyl, substituted alkyl, alkoxy, substituted alkoxy, amino, substituted amino, cyano, carboxyl, carboxyl ester, halo, hydroxy, heterocyclic, substituted hetereocyclic, and nitro; and
n is an integer equal to 0, 1, or 2.

16. A compound of claim 15 wherein R² is cyclohexyl.

17. A compound of claim 16 wherein R³ and R⁴ together with the nitrogen to which they are attached form a morpholino ring.

18. A compound of claim 17 wherein Z is COOH and L is a bond, —CH═CH— or —C(CH₃)═CH—.

19. A compound of claim 18 wherein Y is heteroaryl or substituted heteroaryl.

20. A compound of claim 19 wherein Y is thiazole-5-yl or 2,4-dimethylthiazol-5-yl.

21. The compound selected from the group consisting of
(E)-3-(4-cyclohexyl-5-(2-(2,4-dimethyloxazol-5-yl)quinolin-6-yl)-1-(2-morpholino-2-oxoethyl)-1H-pyrrol-2-yl)acrylic acid;
(E)-3-(5-(2-(5-cyanothiophen-2-yl)quinolin-6-yl)-4-cyclohexyl-1-(2-morpholino-2-oxoethyl)-1H-pyrrol-2-yl)acrylic acid;
(E)-3-(4-cyclohexyl-5-(2-(2,5-dimethylthiazol-4-yl)quinolin-6-yl)-1-(2-morpholino-2-oxoethyl)-1H-pyrrol-2-yl)acrylic acid;
(E)-3-(4-cyclohexyl-5-(2-(3,5-dimethyl-1H-pyrrol-2-yl)quinolin-6-yl)-1-(2-morpholino-2-oxoethyl)-1H-pyrrol-2-yl)acrylic acid;
(E)-3-(4-cyclohexyl-5-(2-(2,4-difluorophenyl)quinolin-6-yl)-1-(2-morpholino-2-oxoethyl)-1H-pyrrol-2-yl)acrylic acid;
(E)-3-(4-cyclohexyl-5-(2-(4-fluorophenyl)quinolin-6-yl)-1-(2-morpholino-2-oxoethyl)-1H-pyrrol-2-yl)acrylic acid;
(E)-3-(4-cyclohexyl-5-(2-(1,3,5-trimethyl-1H-pyrrol-2-yl)quinolin-6-yl)-1-(2-morpholino-2-oxoethyl)-1H-pyrrol-2-yl)acrylic acid;
(E)-3-(4-cyclohexyl-5-(2-(3,5-dimethoxyphenyl)quinolin-6-yl)-1-(2-morpholino-2-oxoethyl)-1H-pyrrol-2-yl)acrylic acid;
(E)-3-(4-cyclohexyl-5-(2-(2-fluorophenyl)quinolin-6-yl)-1-(2-morpholino-2-oxoethyl)-1H-pyrrol-2-yl)acrylic acid;
(E)-3-(4-cyclohexyl-5-(2-(3-methylthiophen-2-yl)quinolin-6-yl)-1-(2-morpholino-2-oxoethyl)-1H-pyrrol-2-yl)acrylic acid;
(E)-3-(5-(2-(3-cyanophenyl)quinolin-6-yl)-4-cyclohexyl-1-(2-morpholino-2-oxoethyl)-1H-pyrrol-2-yl)acrylic acid;
(E)-3-(4-cyclohexyl-5-(2-(4-methylpyridin-2-yl)quinolin-6-yl)-1-(2-morpholino-2-oxoethyl)-1H-pyrrol-2-yl)acrylic acid;
(E)-3-(4-cyclohexyl-1-(2-morpholino-2-oxoethyl)-5-(2-(pyridin-4-yl)quinolin-6-yl)-1H-pyrrol-2-yl)acrylic acid;
(E)-3-(4-cyclohexyl-1-(2-morpholino-2-oxoethyl)-5-(2-p-tolylquinolin-6-yl)-1H-pyrrol-2-yl)acrylic acid;
(E)-3-(4-cyclohexyl-5-(2-(5-ethylthiophen-2-yl)quinolin-6-yl)-1-(2-morpholino-2-oxoethyl)-1H-pyrrol-2-yl)acrylic acid;
(E)-3-(5-(2-(2-amino-4-methylthiazol-5-yl)quinolin-6-yl)-4-cyclohexyl-1-(2-morpholino-2-oxoethyl)-1H-pyrrol-2-yl)acrylic acid;
(E)-3-(4-cyclohexyl-1-(2-morpholino-2-oxoethyl)-5-(2-(N-oxo-pyridin-3-yl)quinolin-6-yl)-1H-pyrrol-2-yl)acrylic acid;
(E)-3-(1-(carboxymethyl)-4-cyclohexyl-5-(2-(2,4-dimethylthiazol-5-yl)quinolin-6-yl)-1H-pyrrol-2-yl)acrylic acid;
(E)-3-(1-((tert-butoxycarbonyl)methyl)-4-cyclohexyl-5-(2-(2,4-dimethylthiazol-5-yl)quinolin-6-yl)-1H-pyrrol-2-yl)acrylic acid;
(E)-3-(4-cyclohexyl-5-(2-(2,4-dimethylthiazol-5-yl)quinolin-6-yl)-1-(2-morpholino-2-oxoethyl)-1H-pyrrol-2-yl)acrylic acid;
(E)-3-(1-(carboxymethyl)-4-cyclohexyl-5-(2-(2,4-dimethylthiazol-5-yl)quinolin-6-yl)-1H-pyrrol-2-yl)-2-methylacrylic acid;
(E)-3-(1-((tert-butoxycarbonyl)methyl)-4-cyclohexyl-5-(2-(2,4-dimethylthiazol-5-yl)quinolin-6-yl)-1H-pyrrol-2-yl)-2-methylacrylic acid;
(E)-3-(4-cyclohexyl-5-(2-(2,4-dimethylthiazol-5-yl)quinolin-6-yl)-1-(2-morpholino-2-oxoethyl)-1H-pyrrol-2-yl)-2-methylacrylic acid;
(E)-3-(4-(carboxymethyl)-1-cyclohexyl-5-(2-(2,4-dimethylthiazol-5-yl)quinolin-6-yl)-1H-pyrrol-3-yl)acrylic acid;
(E)-3-(4-((tert-butoxycarbonyl)methyl)-1-cyclohexyl-5-(2-(2,4-dimethylthiazol-5-yl)quinolin-6-yl)-1H-pyrrol-3-yl)acrylic acid;
(E)-3-(1-cyclohexyl-5-(2-(2,4-dimethylthiazol-5-yl)quinolin-6-yl)-4-(2-morpholino-2-oxoethyl)-1H-pyrrol-3-yl)acrylic acid;
(E)-3-(1-(carboxymethyl)-4-cyclohexyl-5-(2-(2,4-dimethylthiazol-5-yl)quinolin-6-yl)-1H-imidazol-2-yl)acrylic acid;
(E)-3-(1-((tert-butoxycarbonyl)methyl)-4-cyclohexyl-5-(2-(2,4-dimethylthiazol-5-yl)quinolin-6-yl)-1H-imidazol-2-yl)acrylic acid;
(E)-3-(4-cyclohexyl-5-(2-(2,4-dimethylthiazol-5-yl)quinolin-6-yl)-1-(2-morpholino-2-oxoethyl)-1H-imidazol-2-yl)acrylic acid;
1-(carboxymethyl)-4-cyclohexyl-5-(2-(2,4-dimethylthiazol-5-yl)quinolin-6-yl)-1H-pyrrole-2-carboxylic acid;
1-((tert-butoxycarbonyl)methyl)-4-cyclohexyl-5-(2-(2,4-dimethylthiazol-5-yl)quinolin-6-yl)-1H-pyrrole-2-carboxylic acid;
4-cyclohexyl-5-(2-(2,4-dimethylthiazol-5-yl)quinolin-6-yl)-1-(2-morpholino-2-oxoethyl)-1H-pyrrole-2-carboxylic acid;
2-(4-cyclohexyl-5-(2-(2,4-dimethylthiazol-5-yl)quinolin-6-yl)-1-(2-morpholino-2-oxoethyl)-1H-pyrrole-2-carboxamido)acetic acid;
4′-cyclohexyl-5′-[2-(2,4-dimethyl-thiazol-5-yl)-quinolin-6-yl]-1′-(2-morpholin-4-yl-2-oxo-ethyl)-1H,1′H-[2,2′]bipyrrolyl-4-carboxylic acid;
4-cyclohexyl-5-[2-(2,4-dimethyl-thiazol-5-yl)-quinolin-6-yl]-1-(2-morpholin-4-yl-2-oxo-ethyl)-1H,1′H-[2,3′]bipyrrolyl-5′-carboxylic acid;
4′-cyclohexyl-5′-[2-(2,4-dimethyl-thiazol-5-yl)-quinolin-6-yl]-1′-(2-morpholin-4-yl-2-oxo-ethyl)-1H,1′H-[2,2′]bipyrrolyl-5-carboxylic acid;
2-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-1H-imidazole-4-carboxylic acid;
4-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-1H-imidazole-2-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-2H-pyrazole-3-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-2H-[1,2,4]triazole-3-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-furan-3-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-furan-2-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-thiophene-3-carboxylic acid;
5-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-thiophene-2-carboxylic acid;
2-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-oxazole-4-carboxylic acid;
2-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-oxazole-5-carboxylic acid;
2-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-thiazole-4-carboxylic acid; and
2-[4-cyclohexyl-5-[2-(2,4-dimethyl-thiazol-5-yl)-quinolin-6-yl]-1-(2-morpholin-4-yl-2-oxo-ethyl)-1H-pyrrol-2-yl]-thiazole-5-carboxylic acid.

22. A pharmaceutical composition comprising a pharmaceutically acceptable carrier and a therapeutically effective amount of a compound of claim 1 or a mixture of two or more of such compounds.

23. A method for treating or preventing a viral infection in a mammal mediated at least in part by a virus in the Flaviviridae family of viruses which method comprises administering to a mammal a pharmaceutical composition according to claim 22.

24. The method of claim 23 wherein said viral infection is a hepatitis C viral infection.

25. The method of claim 23 in combination with the administration of a therapeutically effective amount of one or more agents active against hepatitis C virus.

26. The method of claim 25 wherein said active agent against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

27. The method of claim 26 wherein said agent active against hepatitis C virus is interferon-alpha or pegylated interferon-alpha alone or in combination with ribavirin or levovirin.
